(19) United States
(12) Patent Application Publication
(10) Pub. No.: US 2023/0355223 A1
(43) Pub. Date: Nov. 9, 2023

(54) METHODS AND APPARATUS FOR CONTROLLED DELIVERY OF A SEALANT

(71) Applicants: J. Christopher Flaherty, Auberndale, FL (US); BetaGlue Technologies S.p.A., Milan (IT); Politecnico di Milano, Milan (IT)

(72) Inventors: J. Christopher Flaherty, Auberndale, FL (US); Antonino Amato, Milan (IT); Jason Schense, Milan (IT); Massimo Romanelli, Milan (IT); Maria L. Constantino, Milan (IT); Francesco de Gaetano, Milan (IT); Kasra Osouli, Milan (IT)

(21) Appl. No.: 17/999,376

(22) PCT Filed: May 20, 2021
(86) PCT No.: PCT/US2021/033507

§ 371 Date: Nov. 18, 2022

Related U.S. Application Data
(60) Provisional application No. 63/027,876, filed on May 20, 2020.

(51) Int. Cl. A61B 17/00; A61M 5/00; A61M 5/145

(52) U.S. Cl. CPC A61B 17/00491; A61M 5/008; A61M 5/14546

(57) ABSTRACT

The present disclosure relates generally to methods and apparatus to control delivery of a sealant during an invasive procedure. Disclosed methods and apparatus control movement of a syringe to control injection of sealant stored therein. For example, disclosed methods and apparatus can displace the syringe body relative to the plunger as the syringe is withdrawn from the target site, rather than displacing the plunger relative to the syringe body, to thereby pressurize the sealant or inject the sealant in an amount proportional to a distance the syringe body is withdrawn. This reduces the variability in the amount of sealant delivered along the syringe withdrawal pathway. The disclosed methods and apparatus can increase precision in movement of a syringe, thereby increasing precision in sealant delivery.

### CROSS-REFERENCE TO RELATED APPLICATIONS

This application claims benefit under 35 U.S.C. § 119(e) to U.S. Provisional Application Ser. No. 63/027,876, filed May 20, 2020, which is hereby incorporated by reference in its entirety.

### TECHNICAL FIELD

The technology described herein relates generally to delivery of a fluid during a surgical procedure, and more specifically to controlled delivery of a sealant during an invasive procedure.

### BACKGROUND

Various medical procedures require access to tissue within the body of a subject, for example, to treat and/or remove target tissue. Such invasive procedures, such as biopsy procedures or locoregional (LR) therapies for example, require instruments to access the target tissue, treat and/or remove the target tissue, and seal any affected tissue. For example, for a biopsy procedure, an introducer, such as a catheter, cannula, sheath, or other tube, may be percutaneously inserted into the patient and guided to the target site. A needle, such as a trocar, may be guided through the introducer to the target site where sample tissue can be excised and removed from the patient by withdrawing the needle back out through the introducer. As another example, for an LR therapy, a delivery device (e.g., delivering drugs or chemicals) or other antitumoral device may be introduced to the target tissue to induce partial or complete necrosis (e.g., of tumor cells). Percutaneous ablation is an exemplary locoregional therapy that involves inserting a needle directly into a tumor under image guidance (e.g., ultrasound or computed tomography) to destroy the tumor through heating, freezing, or the application of a drug or chemical, such as alcohol. When the introducer, delivery device, and/or needle is withdrawn from the subject, a tissue tract is generated along the withdrawal path. The tissue tract and/or excision site can hemorrhage or tumoral seeding can occur if not properly sealed shortly thereafter. In the case of a lung biopsy, improper sealing can result in pneumothorax, or collapsed lung, as air or gas leaks into the membrane lining of the lungs.

To seal affected tissue, such as an excision site and/or tissue tract, resulting from such medical procedures, a sealant fluid can be introduced at the affected site. Current methods to seal affected tissue involve manual use of a syringe to deliver sealant. By these methods, an operator inserts a syringe needle into an introducer while it is still in the subject and injects the sealant into the introducer by pushing, with one hand, the plunger of the syringe distally relative to the syringe body, while pulling, with the other hand, the introducer proximally to withdraw the introducer from the subject. These manual procedures are imprecise and ineffective at delivering an adequate amount of sealant at the appropriate time, resulting in bleeding at the affected site and a higher risk of complications, such as pneumothorax in the case of a lung biopsy.

The information included in this Background section of the specification, including any references cited herein and any description or discussion thereof, is included for technical reference purposes only and is not to be regarded subject matter by which the scope of the invention as defined in the claims is to be bound.

### SUMMARY

Methods and apparatus for controlled delivery of a sealant are disclosed. In one embodiment, an Injection control device is provided, comprising a base member, the base member comprising an elongate body and a rack, a chassis movably coupled to the base member, the chassis comprising a rear chassis movably engaged to the base member and configured to engage a plunger, a front chassis movably engaged to the rear chassis and configured to engage a syringe, and a spiral cam gear assembly interfaced with the rack of the base member and coupled to the chassis. The spiral cam assembly may comprise a spiral cam gear coupled to the rear chassis, the spiral cam gear comprising a plurality of circumferential teeth, a spiral cam recess, and a rotation axle, and a follower pin coupled to the front chassis and located in a spiral cam recess of the spiral cam gear. The spiral cam recess may have a minimum radius and a maximum radius with a radius difference in the range 5 mm to 20 mm. The rear chassis may comprise at least one slot and the front chassis comprises at least one strut slidably located in the at least one slot. The follower pin may be attached to the at least one strut. The front chassis may comprise a syringe cavity configured to engage a syringe. The syringe cavity may comprise a first opening from which a syringe body of a syringe is configured to extend distally, a second opening from which a plunger is configured to extend proximally, and a third opening configured to removably engage a syringe body flange of a syringe. The first opening may be a front opening, the second opening is a rear opening, and the third opening is a top opening. The rear chassis may further comprise a plunger adjustment assembly. The plunger adjustment assembly may comprise a chassis handle movable relative to the rear chassis, and a plunger engagement structure comprising a plunger cavity and configured to be movable relative to the rear chassis and the chassis handle. The plunger cavity may comprise a first opening from which a plunger is configured to extend distally, and a second opening from which the plunger is configured to be removably engaged. The first opening of the plunger cavity may be a front opening, and the second opening of the plunger cavity is a top opening. The plunger engagement structure may further comprise a plunger engagement head in which the plunger cavity resides, and a plunger engagement body with a helical interface. The chassis handle may comprise a helical interface complementary to the helical interface of the plunger engagement body. The plunger engagement body may comprise a helical thread or groove on an outer surface of the plunger engagement body, and the chassis handle may further comprise a lumen containing the helical interface of the chassis handle. The plunger adjustment assembly may further comprise a chassis handle lock extending from the rear chassis and wherein the chassis handle lock is configured to reversibly engage the chassis handle to resist separation of the chassis handle from the rear chassis. The plunger adjustment assembly may further comprise a chassis handle stop configured to resist further rotation of the chassis handle. The plunger engagement structure may be slidably engaged to the rear chassis. The rear chassis may comprise at least one rail and the plunger engagement structure comprises at least one rail attachment that forms a slidable interface with the at least one rail of the rear chassis. The at least one rail may comprise two elongate grooves and the at least one rail attachment may comprise two projections that have complementary mechanical interfit with the two elongate grooves to resist separation of the rear chassis and the plunger engagement structure. The device may also further comprising a main handle projecting from the base member. The base member may comprise a longitudinal recess and the rack is located in the longitudinal recess. The chassis may comprise a bracket engaged to the base member and configured to resist separation of the chassis from the base member.

In another embodiment, a method of using an injection control device may be provided, comprising placing a syringe into an injection control device, rotating a plunger handle of the injection control device to prime the syringe, and holding a body of the injection control device in place while pulling back on the plunger handle of the injection control device to inject a material from the syringe. Placing the syringe into the injection control device may comprise placing a syringe body flange into a syringe body slot of the injection control device, and placing a plunger flange into a plunger slot of the injection control device. The syringe body slot may be located on a movable front chassis of the injection control device and the plunger slot is located on a plunger adjustment structure movably coupled to a rear chassis of the injection control device. Rotating the plunger handle of the injection control device may reduce a distance between the syringe body slot and the plunger slot by moving the plunger adjustment structure relative to the rear chassis. Pulling back on the plunger handle of the injection control device may reduce a distance between the syringe body slot and the plunger slot by moving the front chassis closer to the rear chassis. Pulling back on the plunger handle of the injection control device translates a chassis along the body of the injection control device and reduces a longitudinal length of the chassis as the chassis translates along the body of the injection control device. Pulling back on the plunger handle of the injection control device may rotate a spiral cam gear along a toothed rack of the body of the injection control device. Pulling back on the plunger handle of the injection control device may pull back the syringe body a first pullback distance and pulls back the plunger a second pullback distance, wherein the second pullback distance is less than first pullback distance. A ratio between a first pullback distance interval and a second pullback distance interval may be uniform along the first pullback distance and the second pullback distance. The method may further comprise coupling the syringe to a needle and/or inserting the needle into an injection site. The injection site may be a lung injection site. The needle may be inserted into the injection site before or after the needle is coupled to the syringe. The needle may be inserted into the injection site before or after the syringe is engaged to the injection control device. Rotating the plunger handle may also lock the plunger handle at a rotation stop.

In some embodiments, an injection control device is disclosed. The injection control device includes a two-part base member and a chassis slidably engaged to the base member. The two-part base member includes an interior opening; two parallel rails; and a linear gear rack located in the interior opening. The chassis includes a chassis top, a plunger adjuster, a chassis bottom, and a cam assembly. The chassis top includes a cavity configured to receive a syringe plunger and a syringe body, a proximal handle, two parallel slots, and a proximal plunger adjuster opening. The plunger adjuster includes an enlarged proximal head with a threaded body and a distal end and aligned along the central linear movement axis of the chassis, wherein the threaded body is rotatably engaged to the plunger adjuster opening of the chassis top and configured to extend and withdraw the distal end from the cavity of the chassis top. The chassis bottom is configured to slidably engage the base member along a linear movement range, the chassis bottom including two slots that slidably engage the two parallel rails of the base member, and a circular opening. The cam assembly is rotatably engaged to the circular opening of the chassis bottom, the cam assembly including a lower gear configured to engage and rotate along the linear gear rack; a cam fixedly engaged to the gear, the cam including a cam rotation axis and a cam opening with an arcuate edge and a straight edge, the arcuate edge including a variable radius from the rotation axis, with a radius difference between a smallest radius and a largest radius in the range of 5 mm to 15 mm; and a syringe follower, including two prongs configured to extend from and move along the two parallel slots of the chassis top, and a follower pin that engages the arcuate edge of the cam opening, so that rotation of the cam causes the two prongs to displace from a distal position to a proximal position in the two parallel slots as the follower pin is displaced from contact against the arcuate edge at a location with the largest radius toward a location with the smallest radius.

In some embodiments, an injection control device is disclosed. The injection control device includes a base member and a chassis slidably engaged to the base member. The base member includes an interior opening, two rails, and a linear gear rack located in the interior opening. The chassis is configured to engage a syringe plunger and a syringe body. The chassis includes a proximal handle, and a plunger position adjuster, configured to adjustably displace a syringe plunger; a cam assembly rotatably engaged to the chassis, the cam assembly including: a lower gear configured to engage and rotate along the linear gear rack, and a cam coupled to the gear, the cam comprising a cam rotation axis and a cam opening comprising an arcuate variable radius from the rotation axis; and a syringe follower, configured to engage the syringe body and the cam opening to linearly displace the syringe body relative to the chassis as the syringe follower is displaced as a contact location between the syringe follower and the cam opening changes from a larger radius toward a smaller radius.

In some embodiments, a method for controlled delivery of sealant is disclosed. The method includes engaging a syringe with an injection control device. The syringe includes a syringe body defining a lumen housing sealant, and a plunger positioned at least partially within the lumen for dispensing the sealant. The injection control device includes a base member and a chassis slidably engaged to the base member. The base member includes an interior opening, two rails, and a linear gear rack located in the interior opening. The chassis is configured to engage the plunger and the syringe body. The chassis includes a proximal handle, a plunger position adjuster configured to adjustably displace the plunger, a cam assembly rotatably engaged to the chassis, the cam assembly including a lower gear configured to engage and rotate along the linear gear rack, a cam coupled to the gear, the cam comprising a cam rotation axis and a cam opening comprising an arcuate variable radius from the rotation axis, and a syringe follower, configured to engage the syringe body and the cam opening to linearly displace the syringe body relative to the chassis as the syringe follower is displaced as a contact location between the syringe follower and the cam opening changes from a larger radius toward a smaller radius. The method further includes adjusting the plunger position adjuster to press against the plunger for priming the sealant; pulling the proximal handle to move the chassis in a proximal direction relative to the base member, thereby moving the cam assembly in the proximal direction relative to the base member and along a length of the linear gear rack, wherein the lower gear engages and rotates along the linear gear rack to rotate the cam as the cam assembly moves along the length of the gear rack, and the rotation of the cam linearly displaces the syringe follower in the proximal direction as a contact location between the syringe follower and the cam opening changes from a larger radius toward a smaller radius, thereby linearly displacing the syringe body in the proximal direction relative to the plunger and the chassis; and thereby releasing the sealant as the syringe body pushes the sealant against the plunger, wherein the sealant is released by an amount proportional to a distance the proximal handle is moved.

In some embodiments, an injection control device is disclosed. The injection control device includes a base member, a chassis slidably engaged to the base member, and a syringe body holder slidably engaged to the chassis. The base member includes an interior opening, two parallel base slots, a co-centric gear in the interior opening, and a handle. The co-centric gear includes two lateral gears and a central gear sharing a rotation axis, the lateral gears having a diameter that is smaller than a diameter of the central gear, wherein the co-centric gear is configured to rotate and the lateral gears and the central gear are configured to have the same angular velocity. The chassis includes a cavity configured to receive a syringe plunger and a syringe body holder, two opposing chassis slots defined within the cavity, two parallel chassis rails slidably engaged with the two parallel base slots, two lateral gear racks configured to engage the lateral gears, a proximal plunger adjuster opening, and a plunger adjuster, the plunger adjuster including an enlarged proximal head with a threaded body and a distal end and aligned along the central linear movement axis of the chassis, wherein the threaded body is rotatably engaged to the plunger adjuster opening of the chassis top and configured to extend and withdraw the distal end from the cavity of the chassis top. The syringe body holder includes two parallel holder rails slidably engaged with the two opposing chassis slots, two prongs extending from a top surface of the syringe body holder, and a central gear rack configured to engage the central gear, wherein the central gear rack is configured to move faster relative to the lateral gear racks based on the larger diameter of the central gear relative to the lateral gears, thereby moving the syringe body holder relative to the chassis when the chassis is moved relative to the base member.

In some embodiments, an injection control device is disclosed. The injection control device includes a base member, a chassis slidably engaged to the base member, and a syringe body holder slidably engaged to the chassis. The base member includes an interior opening, two base slots, and a co-centric gear in the interior opening, the co-centric gear including a small gear member and a large gear member, wherein the small gear member has a smaller diameter than the large gear member, and the small gear member and large gear member have the same angular velocity when the co-centric gear rotates. The chassis is configured to engage a syringe plunger and a syringe body holder. The chassis includes a plunger position adjuster, configured to adjustably displace a syringe plunger, and a chassis gear rack configured to engage the small gear member. The syringe body holder is configured to engage a syringe body. The syringe body holder includes a holder gear rack configured to engage the large gear member to displace the syringe body holder relative to the chassis as the holder gear rack moves faster relative to the chassis gear rack based on the larger diameter of the large gear member relative to the small gear member.

In some embodiments, a method for controlled delivery of sealant is disclosed. The method includes engaging a syringe with an injection control device. The syringe includes a syringe body defining a lumen housing sealant, and a plunger positioned at least partially within the lumen for dispensing the sealant. The injection control device includes a base member, a chassis slidably engaged to the base member, and a syringe body holder slidably engaged to the chassis. The base member includes an interior opening, two base slots, and a co-centric gear in the interior opening, the co-centric gear including a small gear member and a large gear member, wherein the small gear member has a smaller diameter than the large gear member, and the small gear member and large gear member have the same angular velocity when the co-centric gear rotates. The chassis is configured to engage a syringe plunger and a syringe body holder. The chassis includes a plunger position adjuster configured to adjustably displace a syringe plunger, and a chassis gear rack configured to engage the small gear member. The syringe body holder is configured to engage a syringe body. The syringe body holder includes a holder gear rack configured to engage the large gear member to displace the syringe body holder relative to the chassis as the holder gear rack moves faster relative to the chassis gear rack based on the larger diameter of the large gear member relative to the small gear member. The method further includes adjusting the plunger position adjuster to press against the plunger for priming the sealant; moving the chassis in a proximal direction relative to the base member, thereby rotating the small gear member as the small gear member engages the chassis gear rack, wherein rotation of the small gear member rotates the large gear member at the same angular velocity and rotation of the large gear member linearly displaces the holder gear rack at a faster speed than the chassis gear rack as the large gear member engages the holder gear rack, thereby displacing the syringe body holder in the proximal direction relative to the chassis and linearly displacing the syringe body in the proximal direction relative to the plunger; and thereby releasing the sealant as the syringe body pushes the sealant against the plunger, wherein the sealant is released by an amount proportional to a distance the chassis is moved.

In some embodiments, a method of collecting tissue from a target location of a patient is disclosed. The method includes selecting a patient and providing a tissue collecting device. The tissue collecting device includes an elongate tube with a proximal portion and a distal portion including a first distal end; a tissue collecting assembly including an elongate portion including a second distal end, wherein the second distal end is configured to pass through the elongate tube and exit the first distal end; treatment material for delivery into the patient; and a material delivery assembly constructed and arranged to deliver the treatment material to a delivery location including one or more anatomical locations of the patient. The method further includes inserting the elongate tube into the patient along an insertion tract; advancing the tissue collecting assembly through the elongate tube and into the target location and to collect a tissue sample; withdrawing the tissue collecting assembly from the patient; delivering the treatment material to the delivery location using the material delivery assembly; and removing the elongate tube from the patient. In some embodiments, a system for collecting tissue is disclosed that uses the tissue collecting device described above.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

FIG. 1 is a perspective view of an injection control device in accordance with one embodiment.

FIG. 2A is a perspective exploded view of the injection control device of FIG. 1.

FIG. 2B is a rear elevation exploded view of the injection control device of FIG. 1.

FIG. 2C is a right side exploded view of the injection control device of FIG. 1.

FIG. 3 is a top plan view of the injection control device of FIG. 1.

FIG. 4 is a rear elevation view of the injection control device of FIG. 1.

FIG. 5 is a right side view of the injection control device of FIG. 1.

FIG. 6 is a cross section view of the injection control device of FIG. 1 taken along line E-E of FIG. 5.

FIG. 7 is a perspective view of a first base member of the injection control device of FIG. 1.

FIG. 8 is a top plan view of the first base member of FIG. 7.

FIG. 9 is a rear elevation view of the first base member of FIG. 7.

FIG. 10 is a right side view of the first base member of FIG. 7.

FIG. 11 is a cross section view of the first base member of FIG. 7 taken along line D-D of FIG. 10.

FIG. 12 is a perspective view of a second base member of the injection control device of FIG. 1.

FIG. 13 is a top plan view of the second base member of FIG. 12.

FIG. 14 is a cross section view of the second base member of FIG. 12 taken along line C-C of FIG. 13.

FIG. 15 is a front elevation view of the second base member of FIG. 12.

FIG. 16 is a right side view of the second base member of FIG. 12.

FIG. 17 is a perspective view of a linear gear rack of the injection control device of FIG. 1.

FIG. 18 is a right side view of the linear gear rack of FIG. 17.

FIG. 19 is a rear elevation view of the linear gear rack of FIG. 17.

FIG. 20 is a perspective view of an upper chassis member of the injection control device of FIG. 1.

FIG. 21 is a top plan view of the upper chassis member of FIG. 20.

FIG. 22 is a front elevation view of the upper chassis member of FIG. 20.

FIG. 23 is a left side view of the upper chassis member of FIG. 20.

FIG. 24 is a perspective view of a plunger adjuster of the injection control device of FIG. 1.

FIG. 25 is a right side view of the plunger adjuster of FIG. 24.

FIG. 26 is a rear elevation view of the plunger adjuster of FIG. 24.

FIG. 27 is a perspective view of a securing mechanism of the injection control device of FIG. 1.

FIG. 28 is a right side view of the securing mechanism of FIG. 27.

FIG. 29 is a top plan view of the securing mechanism of FIG. 27.

FIG. 30 is a cross section view of the securing mechanism of FIG. 27 taken along line E-E of FIG. 29.

FIG. 31 is a perspective view of an upper chassis member of the injection control device of FIG. 1.

FIG. 32 is a top plan view of the upper chassis member of FIG. 31.

FIG. 33 is a rear elevation view of the upper chassis member of FIG. 31.

FIG. 34 is a right side view of the upper chassis member of FIG. 31.

FIG. 35 is a perspective view of a cam of the injection control device of FIG. 1.

FIG. 36 is a top plan view of the cam of FIG. 35.

FIG. 37 is a right side view of the cam of FIG. 35.

FIG. 38 is a perspective view of a cam follower of the injection control device of FIG. 1.

FIG. 39 is a top plan view of the cam follower of FIG. 38.

FIG. 40 is a rear elevation view of the cam follower of FIG. 38.

FIG. 41 is a right side view of the cam follower of FIG. 38.

FIG. 42 is a perspective view of a gear of the injection control device of FIG. 1.

FIG. 43 is a right side view of the gear of FIG. 42.

FIG. 44 is a top plan view of the gear of FIG. 42.

FIG. 45A is a perspective view of the injection control device of FIG. 1 in operation with a syringe.

FIG. 45B is a top plan view of the injection control device of FIG. 45A in a first configuration such that the syringe is in an extended configuration.

FIG. 45C is a top plan view of the injection control device of FIG. 45A in a second configuration such that the syringe is in a retracted configuration.

FIG. 46 is a top plan view of the cam of FIG. 35 and the cam follower of FIG. 38 in operation.

FIG. 47A is a perspective view of an injection control device in a first configuration in accordance with another embodiment.

FIG. 47B is a perspective view of the injection control device of FIG. 47A in a second configuration.

FIG. 48A is a perspective exploded view of the injection control device of FIG. 47A.

FIG. 48B is a top plan exploded view of the injection control device of FIG. 47A.

FIG. 48C is a left side exploded view of the injection control device of FIG. 47A.

FIG. 49 is a left side view of the injection control device of FIG. 47A.

FIG. 50 is a cross section view of the injection control device of FIG. 47A taken along line A-A of FIG. 49.

FIG. 51 is a perspective view of a base housing of the injection control device of FIG. 47A.

FIG. 52 is a top plan view of the base housing of FIG. 51.

FIG. 53 is a left side view of the base housing of FIG. 51.

FIG. 54 is a rear elevation view of the base housing of FIG. 51.

FIG. 55 is a perspective view of a gear axle of the injection control device of FIG. 47A.

FIG. 56 is a right side view of the gear axle of FIG. 55.

FIG. 57 is a rear elevation view of the gear axle of FIG. 55.

FIG. 58 is a perspective view of a securing mechanism of the injection control device of FIG. 47A.

FIG. 59 is a top plan view of the securing mechanism of FIG. 58.

FIG. 60 is a cross section view of the securing mechanism of FIG. 58 taken along line B-B of FIG. 59.

FIG. 61 is a perspective view of a co-centric gear of the injection control device of FIG. 47A.

FIG. 62 is a rear elevation view of the co-centric gear of FIG. 61.

FIG. 63 is a right side view of the co-centric gear of FIG. 61.

FIG. 64 is a perspective view of a chassis body of the injection control device of FIG. 47A.

FIG. 65 is a top plan view of the chassis body of FIG. 64.

FIG. 66 is a right side view of the chassis body of FIG. 64.

FIG. 67 is a front elevation view of the chassis body of FIG. 64.

FIG. 68 is a perspective view of a plunger adjuster of the injection control device of FIG. 47A.

FIG. 69 is a right side view of the plunger adjuster of FIG. 68.

FIG. 70 is a rear elevation view of the plunger adjuster of FIG. 68.

FIG. 71 is a perspective view of a syringe body holder of the injection control device of FIG. 47A.

FIG. 72 is a top plan view of the syringe body holder of FIG. 71.

FIG. 73 is a right side view of the syringe body holder of FIG. 71.

FIG. 74 is a front elevation view of the syringe body holder of FIG. 71.

FIG. 75A is a top plan view of the injection control device of FIG. 47A in operation with a syringe, the injection control device is in a first configuration such that the syringe is in an extended configuration.

FIG. 75B is a top plan view of the injection control device of FIG. 75A in a second configuration such that the syringe is in a retracted configuration.

FIG. 76 is a schematic view of a device for collecting tissue from a target location.

FIG. 77 is a flow chart illustrating a method for collecting target tissue from a target location.

FIGS. 78A to 78C are side, top and frontal views, respectively, of another exemplary embodiment of an injection control device.

FIGS. 79A to 79C are top perspective views of the injection control device of FIGS. 78A to 78C, in initial, primed and retracted configurations, respectively. FIGS. 79D to 79F are corresponding side views, respectively of the injection control device of FIGS. 78A to 78C.

FIGS. 80A and 80B are perspective exploded views of the injection control device of FIGS. 78A to 78C.

FIGS. 81A to 81E are side, top, bottom, frontal and rear views, respectively, of the rear chassis of the injection control device of FIGS. 78A to 78C.

FIGS. 82A to 82E are side, top, bottom, frontal and rear views, respectively, of the front chassis of the injection control device of FIGS. 78A to 78C.

FIGS. 83A and 83B are side and bottom views, respectively, of a spiral cam gear of the injection control device of FIGS. 78A to 78C.

FIGS. 84A to 84C are schematic illustrations of powered injection control devices.

### DETAILED DESCRIPTION

This disclosure is related to methods and apparatus to controllably deliver a viscous fluid. For example, disclosed methods and apparatus can controllably deliver a sealant to a target site in a patient that has undergone a procedure to treat and/or remove tissue at the target site, such as a biopsy procedure or LR therapy. For example, controlled delivery may include controlling a rate, amount, timing, and/or location the fluid is delivered.

In several embodiments, methods and apparatus to control delivery of a sealant during an invasive procedure are disclosed. For example, for a biopsy procedure, an introducer, such as a catheter, cannula, sheath, or other tube can be percutaneously introduced into a patient and positioned proximate a target site. A biopsy device, such as a needle, may be inserted through the introducer and directed to the target site (e.g., lung, liver, kidney, etc.) to remove sample tissue therefrom. After sample tissue is excised, the biopsy device is pulled in a proximal direction away from the excision site and back through the introducer. The introducer may also be withdrawn in the proximal direction, forming a tissue tract in the path of withdrawal. As another example, for an LR therapy, such as percutaneous ablation, a needle may be percutaneously introduced into a patient and directed to a target site, such as a tumor, under image guidance (e.g., ultrasound or computed tomography) to deliver a treatment (e.g., heating, freezing, drug, chemical, etc.). After the treatment is delivered, the needle is pulled in a proximal direction away from the treated site, forming a tissue tract in the path of withdrawal. At substantially the same time as the biopsy device, the introducer, and/or the needle are withdrawn, a sealant can be injected through the introducer to the excision site and/or tissue tract. Disclosed methods and apparatus control the delivery of the sealant such that sealant is delivered at a consistent rate and in an amount that is proportional to a distance the introducer/needle is withdrawn. By increasing control over sealant delivery, disclosed methods and apparatus reduce risks associated with invasive procedures, such as lesions, hemorrhaging, tumoral seeding, pneumothorax, and the like.

In several embodiments, disclosed methods and apparatus control movement of a syringe to control injection of sealant stored therein. For example, disclosed methods and apparatus can displace the syringe body relative to the plunger as the syringe is withdrawn from the target site, rather than displacing the plunger relative to the syringe body, to thereby pressurize the sealant or inject the sealant in an amount proportional to a distance the syringe body is withdrawn. This reduces the variability in the amount of sealant delivered along the syringe withdrawal pathway. The disclosed methods and apparatus can increase precision in movement of a syringe, thereby increasing precision in sealant delivery.

In several embodiments, an apparatus for controlled delivery of a viscous fluid is disclosed. The apparatus can include a chassis that is slidably engaged with a base. The base can include an interior opening, two rails, and a linear gear rack positioned inside the interior opening. The chassis can include a cavity for receiving a syringe plunger and a syringe body, and two slots that slidably engage the two rails of the base. The chassis can also include a plunger position adjuster that is adjustable to engage with or reposition the syringe plunger, for example, to prime fluid stored in the syringe body. The chassis can further include a handle to facilitate movement of the chassis relative to the base by a user.

The disclosed controlled delivery apparatus can include a cam assembly that is rotatably engaged with the chassis. For example, the chassis may include a circular opening on a bottom surface of the chassis that rotatably engages the cam assembly. The cam assembly can include a cam and a lower gear fixedly engaged to the cam and configured to engage and rotate along the linear gear rack of the base. The cam can include a cam rotation axis and a cam opening defining a variable radius from the axis of rotation to the edge of the cam opening. For example, the edge of the cam opening can include an arcuate edge and a straight edge defining the variable radius. As another example, the edge of the cam opening may include varying arcuate edges that define the variable radius (e.g., a different arcuate edge at either end of the straight edge).

The disclosed controlled delivery apparatus can further include a syringe follower configured to engage a syringe body and the cam opening. The syringe follower can be configured to linearly displace the syringe body relative to the chassis as the syringe follower is displaced and a contact location between the syringe follower and the cam opening changes from a larger radius toward a smaller radius. For example, the syringe follower can include two prongs configured to engage the syringe body and a pin configured to engage the cam opening. The two prongs can extend into parallel slots in the chassis to engage the syringe body. The pin can be configured to be displaced against the edge of the cam opening as the cam rotates to linearly displace the prongs and the syringe body.

In several embodiments, disclosed methods include moving the chassis in a proximal direction (e.g., towards the handle or towards a user). For example, when the chassis is moved in a proximal direction, the cam is linearly displaced along a length of the linear gear rack, rotating the gear as the gear engages the linear gear rack, and thereby rotating the cam. When the cam rotates, the contact point between the syringe follower and the cam opening edge changes, changing the radius from a larger to a smaller radius, resulting in linear displacement of the syringe follower in the proximal direction. When the syringe follower engages a syringe body, the syringe body is also linearly displaced in the proximal direction. The plunger position adjuster engages the syringe plunger and keeps the syringe plunger fixed as the syringe body moves relative to the syringe plunger. When a viscous fluid, such as sealant, is housed inside the syringe body, the movement of the syringe body relative to the syringe plunger pushes the fluid against the plunger, thereby pushing the fluid out the distal tip of the syringe body and delivering the fluid in a controlled manner. For example, an amount of fluid delivered is proportional to a distance the syringe body is displaced.

The linear displacement of the syringe follower can be in the same direction as the chassis. A distance the syringe follower moves relative to the chassis can be proportional to a distance the chassis moves relative to the base. Further, because the syringe follower moves relative to the chassis and the chassis further moves relative to the base, the syringe follower moves a greater distance relative to the base than the distance the chassis is moved relative to the base. In other words, movement of the chassis across a small distance is translated to greater total movement of the syringe follower, thereby providing a mechanical advantage that is not found in existing delivery systems.

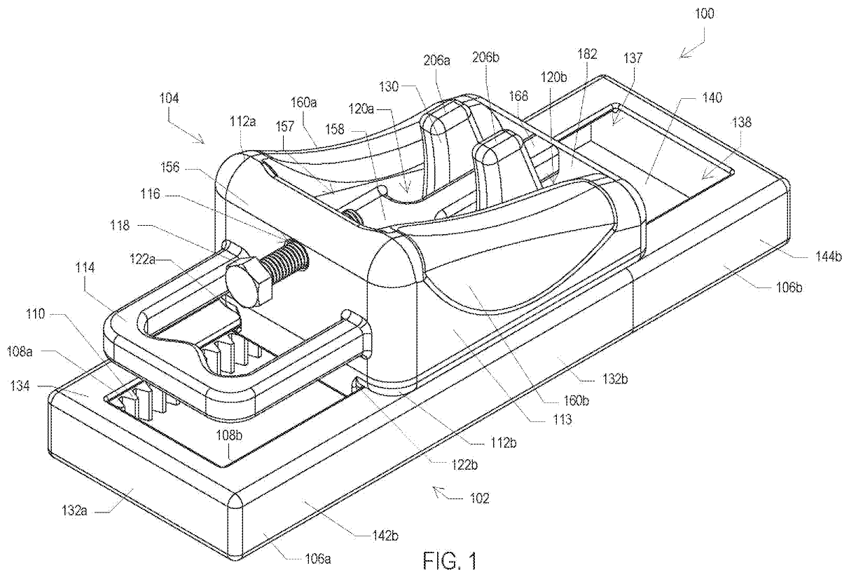

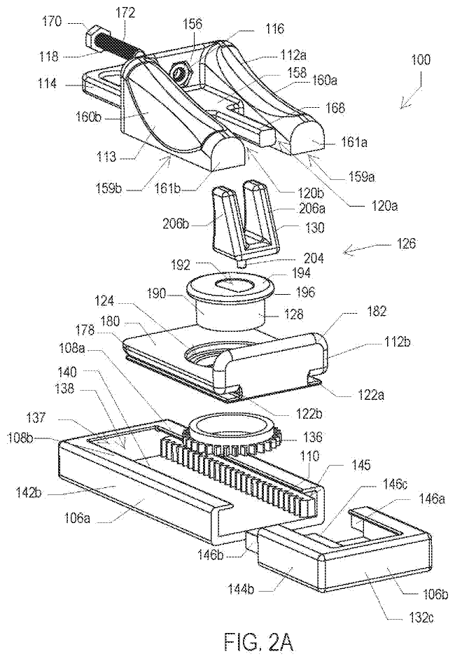

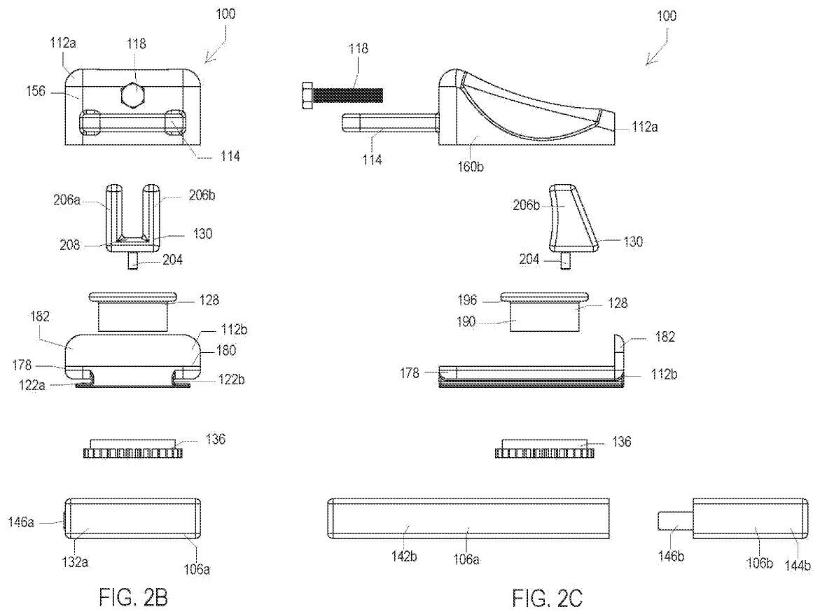

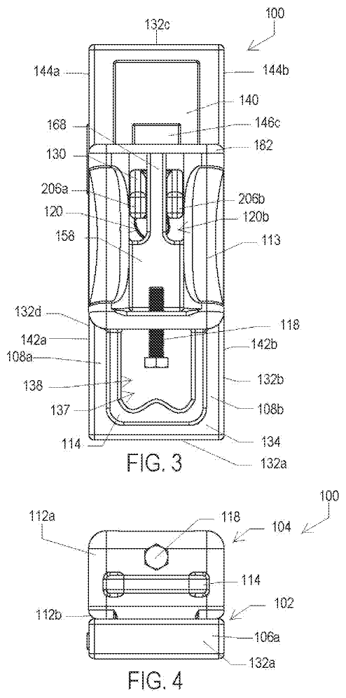

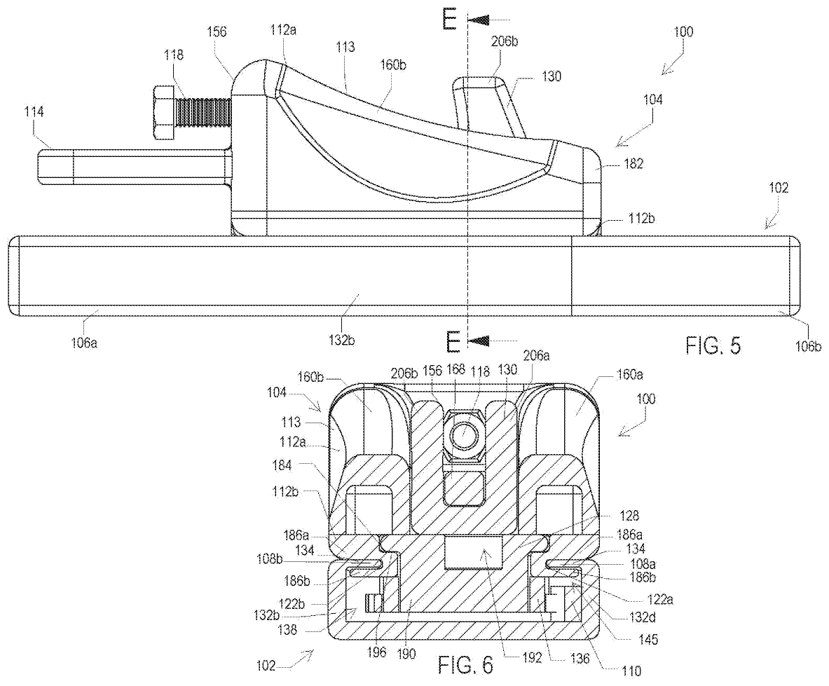

Turning to the figures, FIG. 1 illustrates an apparatus for controlled delivery or injection of a fluid. FIG. 1 is a perspective view of an injection control device 100 in accordance with one embodiment. FIG. 2A is a perspective exploded view of the injection control device 100 of FIG. 1, FIG. 2B is a rear exploded view of the injection control device 100 of FIG. 1, and FIG. 2C is a right side exploded view of the injection control device 100 of FIG. 1. FIG. 3 is a top plan view of the injection control device 100 of FIG. 1, FIG. 4 is a rear elevation view of the injection control device 100 of FIG. 1, and FIG. 5 is a right side view of the injection control device 100 of FIG. 1. FIG. 6 is a cross section view of the injection control device 100 of FIG. 1 taken along line E-E of FIG. 5. As shown in FIGS. 1-5, the injection control device 100 may include a base assembly or member 102 and a chassis assembly or member 104. The base assembly 102 may include two base members 106a,b, two parallel rails 108a,b, and a linear gear rack 110. The chassis assembly 104 may include an upper or top chassis member 112a, a lower or bottom chassis member 112b, a handle 114, a plunger adjuster opening 116, a plunger adjuster or plunger position adjuster 118, two parallel upper chassis member slots 120a,b, opposing bottom chassis member slots 122a,b, and a cam follower assembly 126. The cam follower assembly 126 may include a cam 128, a cam or syringe follower 130, and a gear 136. As shown, the chassis assembly 104 is slidably engaged with the base assembly 102 by the bottom chassis member slots 122a,b engaging with the parallel rails 108a,b of the base assembly 102.

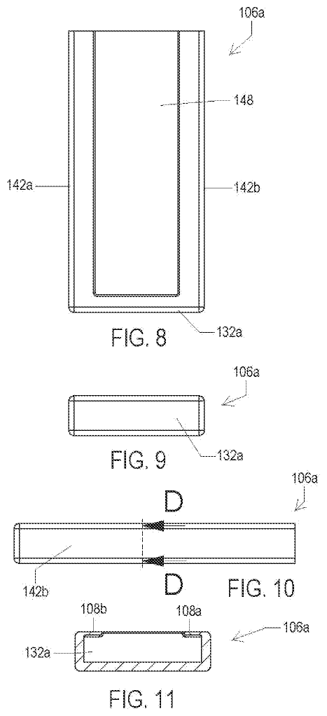

With reference to FIGS. 2A, 3, and 7-19, the base assembly 102 will now be discussed in more detail. As shown, the base assembly 102 includes sidewalls 132a,b,c,d. As shown, the sidewalls 132a,b,c,d are perpendicular to one another with a first sidewall 132a perpendicular to a second sidewall 132b and opposing a third sidewall 132c. A fourth sidewall 132d opposes the second sidewall 132b and is also perpendicular to the first and third sidewalls 132a,c. As shown, the sidewalls 132a,b,c,d form a rectangular shape of the base assembly 102. The base assembly 102 may also include a top wall 134. As shown, the top wall 134 is coupled to the sidewalls 132a,b,c,d. However, it is contemplated that the top wall 134 is only coupled to opposing second and fourth sidewalls 132b,d. In this example, the top wall 134 may be two separate components coupled to each of the second and fourth sidewalls 132b,d. As shown, the top wall 134 includes an interior opening 137 to a cavity 138 that is defined at least in part by the sidewalls 132a,b,c,d and the top wall 134. The cavity 138 may be further defined by an inner bottom wall 140 defining the bottom of the cavity 138. The interior opening 137 forms a gap or space between portions of the top wall 134 and defines two parallel rails 108a,b, the first rail 108a coupled to the fourth sidewall 132d and the second rail 108b coupled to the second sidewall 132b. While the parallel rails 108a,b are depicted as part of the top wall 134, it is contemplated that the parallel rails 108a,b may be positioned anywhere along the height of the second and fourth sidewalls 132b,d. While the parallel rails 108a,b are depicted as flat walls, it is contemplated that the parallel rails 108a,b may be round, for example a rod or wire, or rectangular, or another shape configured to fit in the opposing bottom chassis member slots 122a,b, as discussed in more detail below. Alternatively, the parallel rails 108a,b may be slots, for example where the opposing bottom chassis member slots are rails, such that the rails of the chassis assembly 104 fit within the slots of the base assembly 102 to slidably engage the chassis assembly 104 to the base assembly 102.

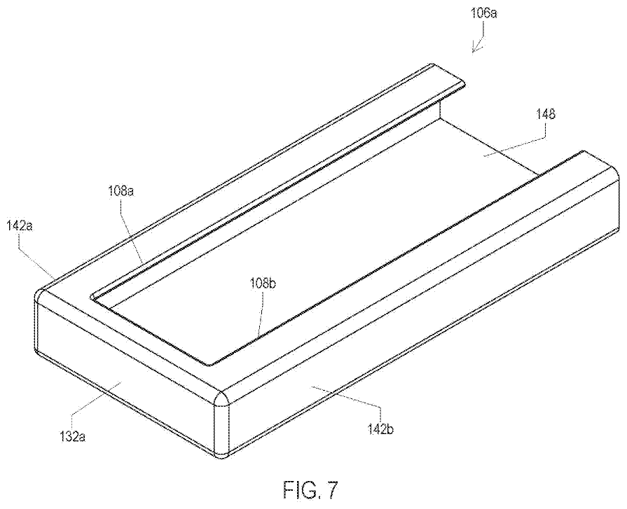

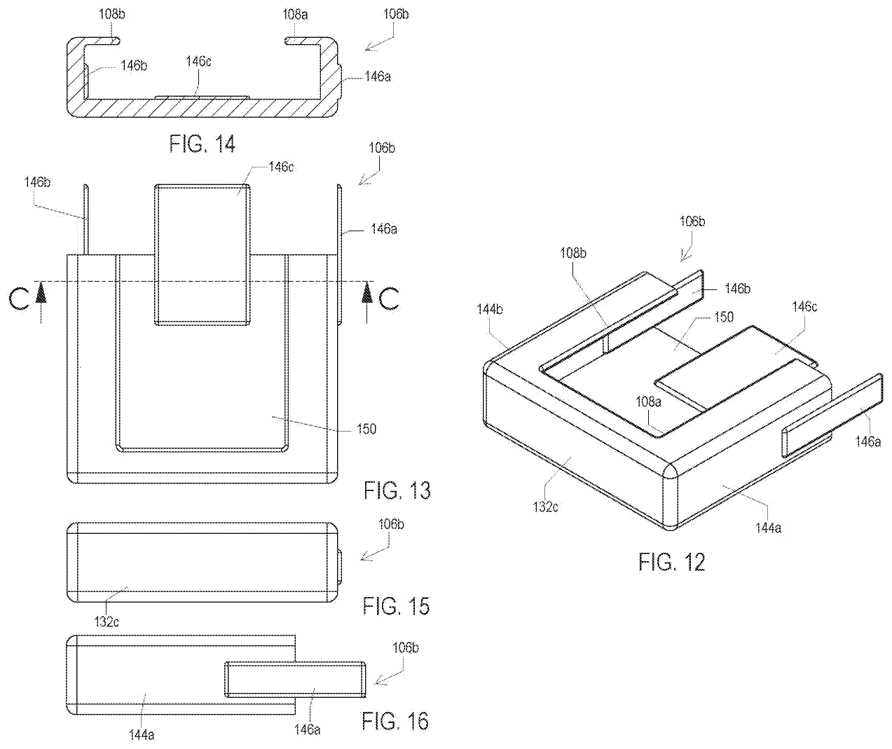

As shown, the base assembly 102 includes two base members 106a,b coupled together to form the sidewalls 132a,b,c,d, top wall 134, inner bottom wall 140 and cavity 138. As shown in FIGS. 7-11, the first base member 106a includes the first sidewall 132a, first base member first and second opposing sidewalls 142a,b that form part of opposing fourth and second sidewalls 132d,b, respectively, and a first base member inner bottom wall 148 that forms part of the inner bottom wall 140. As shown in FIGS. 12-16, the second base member 106b includes the third sidewall 132c, second base member first and second opposing sidewalls 144a,b that form the other part of opposing fourth and second sidewalls 132d,b, respectively, and a second base member inner bottom wall 150 that forms the other part of the inner bottom wall 140. In the depicted embodiment, the second base member 106b is smaller in length than the first base member 106a; however, it is contemplated that the second base member 106b may have the same or greater length than the first base member 106a.

The second base member 106b includes tabs 146a,b,c. As shown, tabs 146a,b,c are flat panels. As shown, the first and second tabs 146a,b are coupled to the second base member first and second opposing sidewalls 144a,b, respectively. The first tab 146a is coupled to an outer surface of the second base member first sidewall 144a, and the second tab 146b is coupled to an inner surface of the second base member second sidewall 144b; however, it is contemplated that the first and second tabs 146a,b may be coupled on either side of either wall, in any configuration. The third tab 146c is coupled to the second base member inner bottom wall 150. While the figures depict the tabs 146a,b,c coupled to the second base member 106b, it is contemplated that the tabs may be coupled to the first base member 106a. The tabs 146a,b,c may help to provide a greater surface area of overlap between the base members 106a,b and/or can assist with aligning the base members 106a,b to attach the base members 106a,b together. While the tabs 146a,b,c are depicted as separate components coupled to the second base member 106b, it is contemplated the tabs 146a,b,c may be integral components (for example, extensions) of the respective first and second opposing sidewalls 144a,b and inner bottom wall 150. The first and second tabs 146a,b may engage the first base member first sidewall 142a and the first base member second sidewall 142b, respectively, and the third tab 146c may engage the first base member inner bottom wall 148, when the first and second base members 106a,b are coupled together to form the base assembly 102. It is contemplated that the first and second base members 106a,b may be coupled by one or more conventional fastening means, such as, for example, adhesive, heat melding, solvent bonding, UV bonding, ultrasonic welding, mechanical fasteners (screws, rivets, etc.), mechanical snap fits, and the like. While the figures depict the base assembly 102 with two base members 106a,b, it is contemplated that the base assembly 102 may be comprised of a single member or comprised of more than two members.

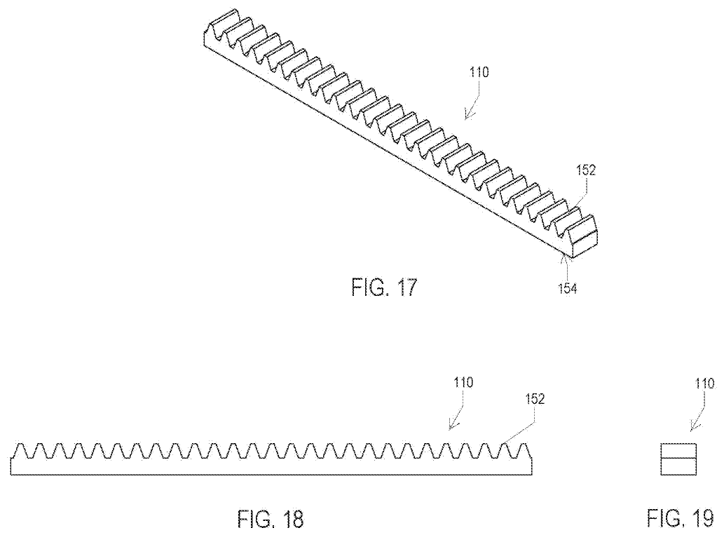

The base assembly 102 may include a linear gear rack 110. The linear gear rack 110 may include a plurality of gear rack teeth 152 arranged in parallel along a length of the linear gear rack 110. As shown in FIGS. 17-19, the linear gear rack 110 may have a rectangular shape. For example, the linear gear rack 110 may have a bottom surface 154 that is rectangular in shape. As shown in FIG. 2A, the linear gear rack 110 may be sized to extend along the entire length of the first base member 106a, such that the linear gear rack 110 does not extend into the second base member 106b; however, it is contemplated that the linear gear rack 110 may have a length that is greater than the length of the first base member 106a, such that the linear gear rack 110 extends into the second base member 106b. It is also contemplated that the linear gear rack 110 may be positioned entirely or partially within the second base member 106b.

In the assembled configuration, as shown in FIGS. 1 and 2A for example, the linear gear rack 110 may be positioned within the cavity 138 of the base assembly 102. For example, the linear gear rack 110 may be positioned along one of the opposing sidewalls 132b,d. In the example depicted, the linear gear rack 110 is positioned along the fourth sidewall 132d. For example, the linear gear rack 110 is positioned such that the bottom surface 154 engages a surface of the fourth sidewall 132d and the plurality of gear rack teeth 152 are arranged in a perpendicular orientation to the inner bottom wall 140 of the cavity 138. The linear gear rack 110 may be coupled to the fourth sidewall 132d by one or more conventional fastening means, such as for example, adhesive, heat melding, solvent bonding, UV bonding, ultrasonic welding, mechanical fasteners (screws, rivets, etc.), mechanical snap fits, and the like. The linear gear rack 110 may be positioned along any length of the base assembly 102. As shown in FIG. 2A, the linear gear rack 110 is positioned along a length of the base assembly 102 corresponding to the length of the first base member first sidewall 142a. As shown, the linear gear rack 110 is positioned such that there is a gap 145 between the linear gear rack 110 and the first rail 108a. The gap 145 is sized to allow the first bottom chassis member slot 122a to slidably engage the first rail 108a, as discussed in more detail below.

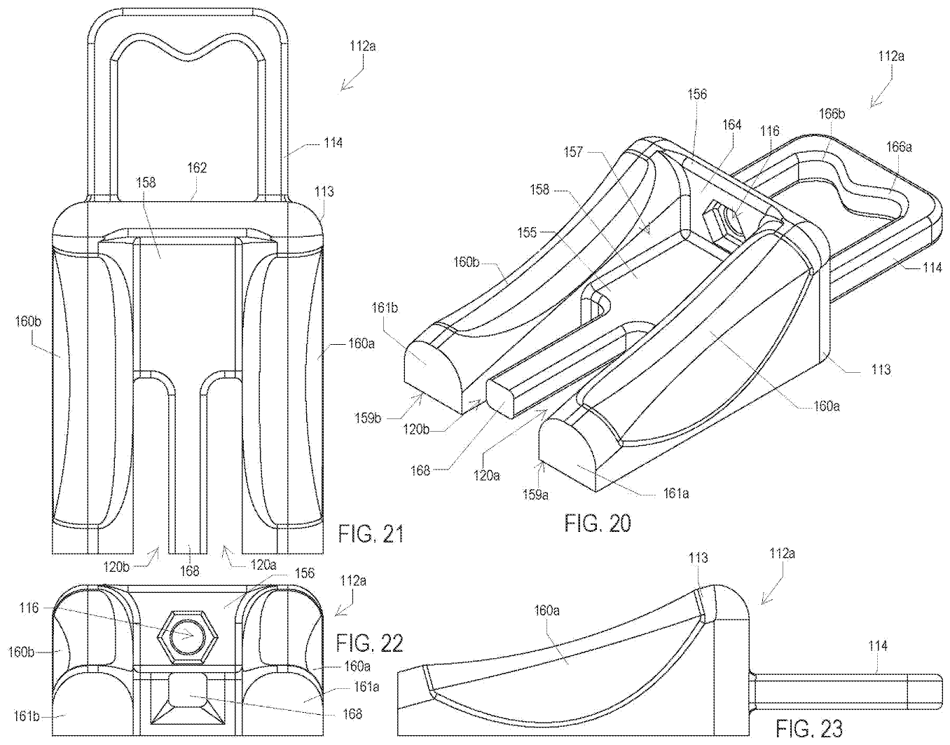

With reference to FIGS. 2A-C, and 20-44, the chassis assembly 104 will now be discussed in more detail. As discussed, the chassis assembly 104 may include an upper or top chassis member 112a, a lower or bottom chassis member 112b, and a cam assembly 126. As shown in FIGS. 2A-C and 21-30, the top chassis member 112a may include a top chassis member body 113, a handle 114, a plunger adjuster opening 116, a plunger adjuster 118, and two parallel top chassis member slots 120a,b. The top chassis member body 113 may include a proximal wall 156 extending from a top chassis member bottom wall 158, and opposing first and second top chassis member sidewalls 160a,b extending from the proximal wall 156 in a distal direction and on opposing sides of the top chassis member bottom wall 158. The proximal wall 156, top chassis member bottom wall 158 and opposing first and second top chassis member sidewalls 160a,b form a chassis cavity 157 for receiving a syringe body and syringe plunger. The proximal wall 156 may include a proximal surface 162 and distal surface 164. The proximal wall 156 may define a plunger adjuster opening 116 therethrough. As shown in FIG. 20, the plunger adjuster opening 116 may be shaped to receive the plunger adjuster 118 and/or securing mechanism 174. For example, the plunger adjuster opening 116 may have a hexagonal shape on the distal surface 164 of the proximal wall 156 to receive a hexagonal shaped securing mechanism 174. While the plunger adjuster opening 116 depicted does not include threading, it is contemplated that the plunger adjuster opening 116 may be threaded.

The handle 114 may be coupled to the top chassis member 112a on the proximal surface 162 of the proximal wall 156. As shown, the handle 114 forms a loop having two indentations 166a,b on a proximal end of the handle 114, for example, for a user's fingers to grasp. However, any ergonomic shape is contemplated for the handle 114 that enables a user to grasp the handle 114 with a hand or one or more fingers. For example, the handle 114 may have a hook shape instead of a loop shape. As shown, the handle 114 is coupled to the proximal wall 156 below the plunger adjuster opening 116; however, it is contemplated that the handle 114 may be coupled to the proximal wall 156 in any manner that does not interfere with access to the plunger adjuster opening 116, for example, above the plunger adjuster opening 116. As shown, the handle 114 is positioned in a horizontal orientation; however a vertical orientation is also contemplated.

The top chassis member bottom wall 158 may define one or more slots. For example, as shown, the top chassis member bottom wall 158 defines two parallel top chassis member slots 120a,b. The top chassis member slots 120a,b are separated by a divider 168. However, it is contemplated that the top chassis member bottom wall 158 may include more or less than two top chassis member slots, for example a single slot or an aperture may be defined in the top chassis member bottom wall 158. As shown, the opposing first and second top chassis member sidewalls 160a,b are positioned on opposing sides of the top chassis member bottom wall 158 and are angled or slant downward from the proximal wall 156 to the distal end of the top chassis member 112a. However, other shapes are contemplated for the first and second top chassis member sidewalls 160a,b, such as, for example, a rectangular or planar shape. The first and second top chassis member sidewalls 160a,b include corresponding first and second top chassis member distal surfaces 161a,b disposed at a distal end of the top chassis member 112a. As shown, the first and second top chassis member distal surfaces 161a,b are parallel to the distal surface 164 of the proximal wall 156. The first and second top chassis member sidewalls 160a,b further include corresponding first and second top chassis member bottom surfaces 159a,b.

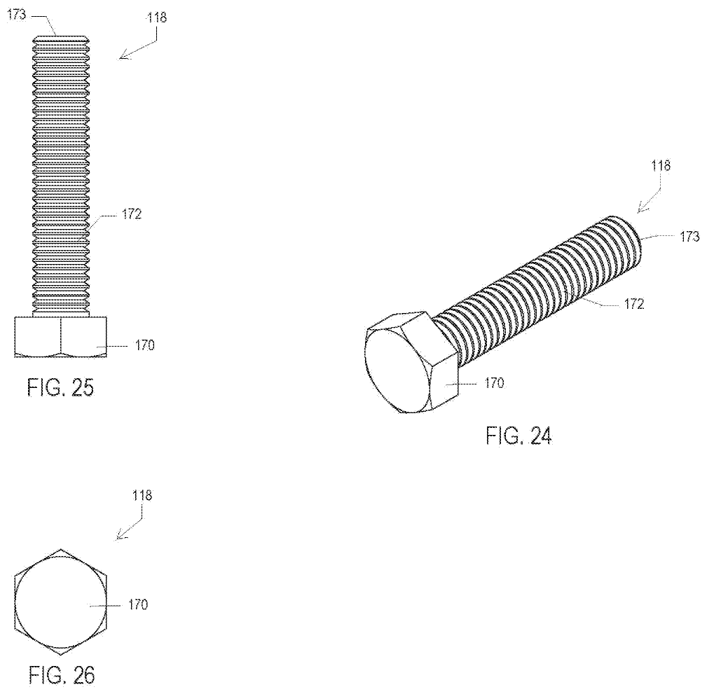

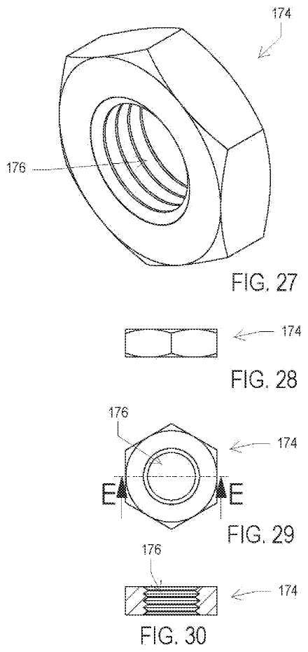

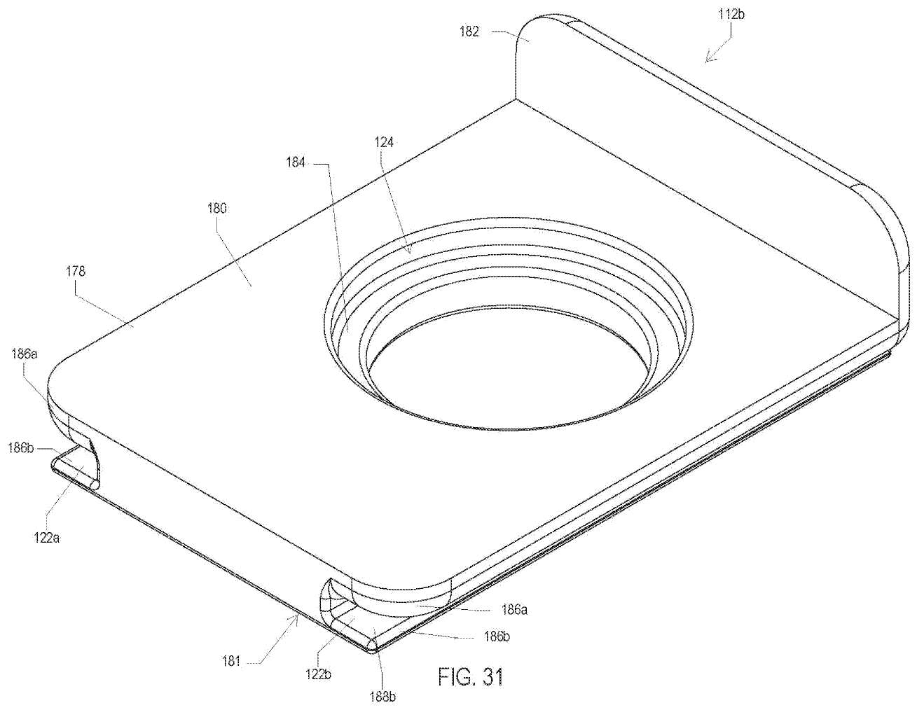

As shown in in FIGS. 1-5, the plunger adjuster 118 may be rotatably engaged to the plunger adjuster opening 116 on the proximal wall 156 and aligned along a central linear movement axis of the top chassis member 112a. FIGS. 24-30 show an exemplary plunger adjuster 118. The depicted plunger adjuster 118 has an enlarged proximal head 170 with a threaded body 172 having a distal threaded body end 173. As shown, the enlarged proximal head 170 has a hexagonal shape; however, other shapes are contemplated, for example, a circular shape, square shape, a T-handle or L-handle shape. As one example, the plunger adjuster 118 may be a bolt, screw, or other similar fastener. As shown in FIGS. 27-30, the plunger adjuster 118 may include a securing mechanism 174. As shown, the securing mechanism 174 may include a threaded aperture 176 for receiving the threaded body 172. The securing mechanism 174 depicted has a hexagonal shape, for example, to correspond with the hexagonal shape on the distal surface 164 of the proximal wall 156 of the top chassis member 112a. As one example, the securing mechanism 174 may be a nut or other similar fastener. The securing mechanism 174 may be rotatably engaged to the threaded body 172 of the plunger adjuster 118 on the distal side of the proximal wall 156 to prevent the plunger adjuster 118 from moving. However, it is also contemplated that the securing mechanism 174 may be omitted, for example where the plunger adjuster 118 is configured to stay in place within the plunger adjuster opening 116, e.g., where the plunger adjuster opening 116 is threaded.

As shown in FIGS. 2A-C and 31-34, the bottom chassis member 112b may include a bottom chassis member body 178, opposing bottom chassis member slots 122a,b and a circular opening 124. The bottom chassis member body 178 may include a bottom chassis member top surface 180 and a bottom chassis member distal wall 182. The bottom chassis member distal wall 182 may extend from the bottom chassis member top surface 180, for example, at a perpendicular angle relative to the bottom chassis member top surface 180. The circular opening 124 may be defined in the bottom chassis member body 178, for example within the bottom chassis member top surface 180. The circular opening 124 may have a securing means 184 disposed about a circumference of the circular opening 124. For example, the securing means 184 may be threading or a lip or ridge disposed about the circumference of the circular opening 124.

The opposing bottom chassis member slots 122a,b may be defined on opposing lateral sides of the bottom chassis member body 178. The bottom chassis member slots 122a,b may have an upper slot member 186a defining an upper slot surface 188a and a lower slot member 186b defining a lower slot surface 188b. The upper slot member 186a has a thickness defined by a distance between the bottom chassis member top surface 180 and the upper slot surface 188a and the lower slot member 186b has a thickness defined by a distance between the lower slot surface 188b and the bottom chassis member bottom surface 181. As shown in FIG. 33, the upper slot member 186a is thicker than the lower slot member 186b; however, it is contemplated that the upper and lower slot member 186a,b may have the same or varying thicknesses. As shown, the upper slot member 186a extends in a lateral direction beyond the lower slot member 186b, such that the bottom chassis member top surface 180 has a greater width between lateral sides of the bottom chassis member body 178 than the width of the bottom chassis member bottom surface 181. The size and shape of the opposing bottom chassis member slots 122a,b may be selected to correspond to the shape of the parallel rails 108a,b of the base assembly. It is further contemplated that the opposing bottom chassis member slots 122a,b may be rails, for example, where the parallel rails 108a,b of the base assembly 102 are slots, to engage with the slots of the base assembly 102.

As shown in FIGS. 2A-C and 35-44, the cam assembly 126 may include a cam 128, a cam or syringe follower 130, and a gear 136. The cam 128 may include a cylindrical body 190 and a cam opening 192. The cam opening 192 may be defined within a top cam surface 194. The top cam surface 194 may be a circular surface having a diameter greater than a diameter of the cylindrical body 190, forming a rim 196 about the circumference of the cylindrical body 190. The cam opening 192 may include an arcuate edge 198 and a straight edge 200. The cam 128 may include an axis of rotation R about which the cam 128 rotates. A radius r may be defined between the axis of rotation R and the arcuate edge 198. The radius r may vary about the circumference of the cam opening 192.

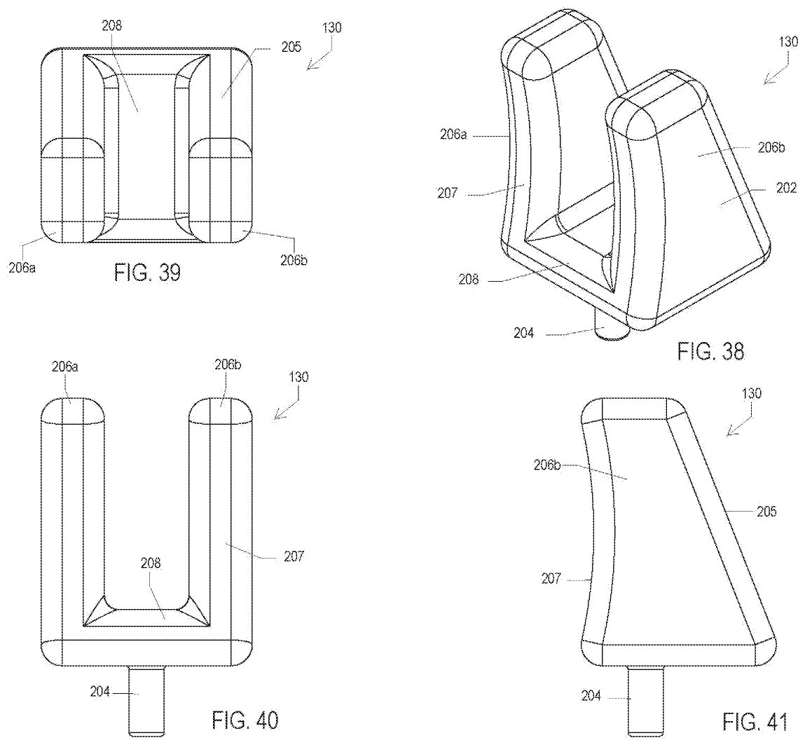

As shown in FIGS. 38-41, the cam or syringe follower 130 may include a follower body 202 and a follower pin 204. As shown, the follower body 202 includes a proximal surface 207 and a distal surface 205. As shown in FIG. 41, the proximal surface 207 may have slight curvature; however, it is also contemplated that the proximal surface 207 may be flat or slanted. As shown, the distal surface 205 is substantially slanted, extending in an outward direction away from the proximal surface 207 along a length of the distal surface 205 towards a follower base 208. It is contemplated, however, that the distal surface 205 may be substantially flat and perpendicular to the follower base 208 or have slight curvature similar to proximal surface 207. The follower body 202 may include two prongs 206a,b extending from the follower base 208. For example, the prongs 206a,b and follower base 208 may form a U-shaped structure. The prongs 206a,b are configured to engage a syringe body; however, other structural features are contemplated to engage a syringe body, such as, for example a ridge, lip or hook extending up from and movably engaged to the top chassis member bottom wall 158, or a structure having an opening to latch a part of the syringe body, the structure movably engaged to the top chassis member bottom wall 158. While two prongs 206a,b are depicted, greater or fewer prongs are also contemplated. The follower pin 204 may extend from the follower base 208 in a direction opposite the prongs 206a,b. As shown, the follower pin 204 is cylindrical.

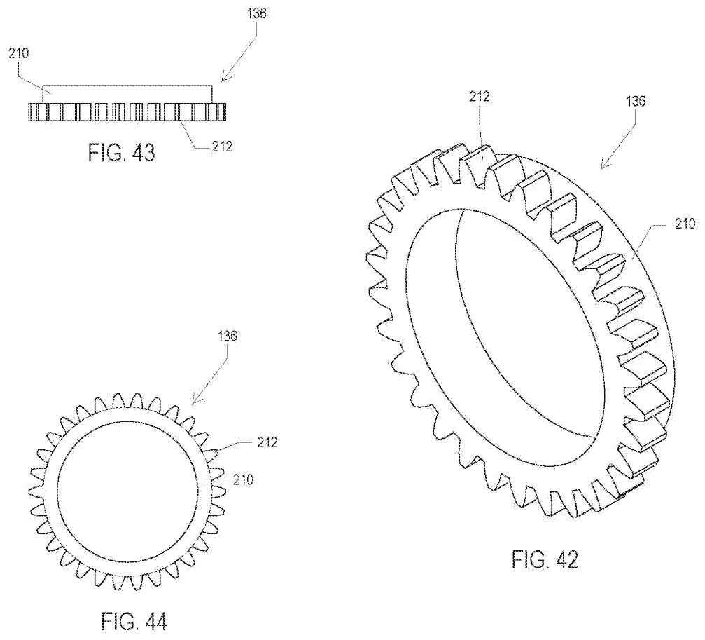

As shown in FIGS. 42-44, the gear 136 may be a gear ring 210 having a plurality of gear teeth 212 disposed about an outer circumference of the gear ring 210. The gear ring 210 may be sized to correspond with a size of the cylindrical body 190 of the cam 128. It is contemplated that the gear 136 may be integral with the cam 128, for example a plurality of gear teeth may be disposed about a circumference of the cylindrical body 190.

An assembled injection control device 100 will now be discussed in more detail. For example, an assembled chassis assembly 104 may include the upper or top chassis member 112a fixedly coupled to the lower or bottom chassis member 112b with the cam assembly 126 extending at least partially therethrough. For example, the top chassis member 112a may be positioned relative to the bottom chassis member 112b such that the first and second top chassis member bottom surfaces 159a,b of the respective first and second top chassis member sidewalls 160a,b engage with the bottom chassis member top surface 180. The first and second top chassis member distal surfaces 161a,b may engage with the bottom chassis member distal wall 182. The top chassis member 112a may be positioned above the bottom chassis member 112b such that the two parallel upper chassis member slots 120a,b align at least partially with the circular opening 124 of the bottom chassis member 112b, defining a continuous opening from a top surface 155 of the top chassis member bottom wall 158 to the bottom chassis member bottom surface 181. The top chassis member 112a may be coupled to the bottom chassis member 112b by one or more conventional fastening means, such as, for example, adhesive, heat melding, solvent bonding, UV bonding, ultrasonic welding, mechanical fasteners (screws, rivets, etc.), mechanical snap fits, and the like.

The cam assembly 126 may be positioned at least partially within the upper chassis member slots 120a,b and at least partially within the circular opening 124 of the bottom chassis member 112b. For example, the cam 128 may be positioned at least partially within the circular opening 124 and rotatably engages the circular opening 124. For example, at least part of the cylindrical body 190 may be positioned within the circular opening. The cam 128 may include an engagement feature that engages with the circular opening 124 to hold the cam 128 vertically in place, such that the cam 128 does not fall through the circular opening 124. For example, the engagement feature can be configured to engage the securing means 184 disposed about the circumference of the circular opening 124 to hold the cam 128 vertically in place. For example, the engagement feature may be the rim 196 disposed about the circumference of the cylindrical body 190. The rim 196 may engage the securing means 184. For example, where the securing means 184 is a lip, the rim 196 may rest above the lip to hold the cam 128 vertically in place. The cam 128 may be positioned vertically within the circular opening 124 such that at least a portion of the cylindrical body 190 is positioned below the bottom chassis member bottom surface 181.

The cam assembly 126 may also be positioned at least partially within the upper chassis member slots 120a,b. For example, at least part of the cam follower 130 may extend into the upper chassis member slots 120a,b. For example, as shown in FIG. 1, the prongs 206a,b extend through and are slidable engaged with the upper chassis member slots 120a,b. The follower pin 204 is positioned at least partially within the cam opening 192 and contacts the arcuate edge 198 and/or straight edge 200. The distance between the point of contact between the follower pin 204 and the arcuate edge 198 and/or straight edge 200 and the axis of rotation R defines the radius r.

The gear 136 may be positioned about a circumference of the cam 128. For example, the gear 136 may be disposed about a circumference of the cylindrical body 190, for example, on a lower end of the cylindrical body 190.

As shown in FIG. 1, the assembled chassis assembly 104 may be slidably engaged with the base assembly 102. For example, the chassis assembly 104 may be positioned within the interior opening 137 to the cavity 138. The bottom chassis member slots 122a,b may be slidably engaged with the parallel rails 108a,b, of the base assembly 102. For example, as shown in FIG. 6, the top wall 134 of the base assembly 102 may be positioned between the upper and lower slot members 186a,b. The lower slot member 186b of the first bottom chassis member slot 122a is positioned between the top wall 134 and the linear gear rack 110, within the gap 145 defined therebetween; however, it is also contemplated that the lower slot member 186b of the second bottom chassis member slot 122b may be positioned between the top wall 134 and the linear gear rack 110 when the linear gear rack 110 is positioned adjacent the second sidewall 132b.

The gear 136 may be positioned between the bottom chassis member bottom surface 181 and inner bottom wall 140 of the base assembly 102, for example within the cavity 138. The gear 136 may be positioned adjacent the linear gear rack 110. For example, one or more of the plurality of gear teeth 212 may engage one or more of the plurality of gear rack teeth 152. The portion of the cylindrical body 190 extending below the bottom chassis member bottom surface 181 may also be positioned within the cavity 138.

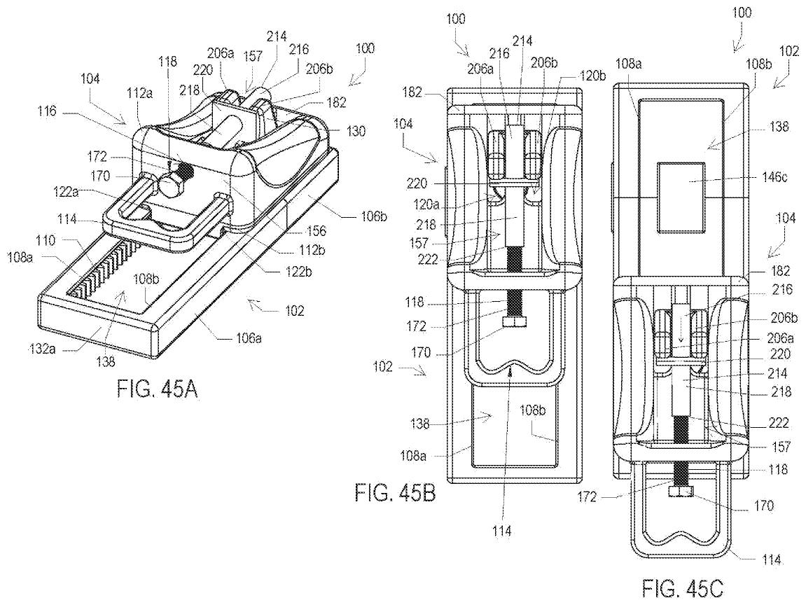

In operation, a delivery device, such as a syringe, may be placed within the chassis cavity 157. For example, FIGS. 45A-45C show the injection control device 100 being used with an exemplary syringe 214. As shown, the exemplary syringe 214 includes a syringe body 216 defining a lumen and a plunger 218 positioned at least partially within the lumen and movably coupled to the syringe body 216. The syringe body 216 may include a flange 220 at a proximal end of the syringe body 216 and a dispense or delivery tip at a distal end of the syringe body 216. The plunger 218 may include a plunger surface 222 at a proximal end that is shaped to allow a user to press on the plunger 218, for example a flat surface. It is contemplated that the syringe may be a conventional syringe, including, for example, a double barrel syringe housing fluid in two separate chambers. The syringe body lumen may house one or more viscous fluids, for example, a sealant. The sealant may be any biocompatible material or bio glue used to seal lesions or excision sites in a patient, including, for example a hemostatic material or procoagulant, fibrin glue, collagen, polyethylene glycol, hydrogels, hyaluronic acid, polylactic acid, and the like. The plunger 218 is configured to press against the fluid stored within the syringe body lumen when the plunger 218 and syringe body 216 are moved relative to one another to push the fluid out of the delivery tip.

In several embodiments, the syringe plunger 218 may be positioned proximate a proximal end of the top chassis member 112a, e.g., adjacent the proximal wall 156, and the syringe body 216 may be positioned proximate a distal end of the top chassis member 112a, e.g., proximate the first and second top chassis member distal surfaces 161a,b and/or the upper chassis member slots 120a,b. For example, the syringe body 216 may be positioned between the prongs 206a,b of the syringe follower 130 extending through the upper chassis member slots 120a,b. The syringe body 216 may be positioned such that the flange 220 at the proximal end is positioned on a proximal side of the prongs 206a,b, for example, engaging the proximal surface 207 of the prongs 206a,b. The plunger 218 may be positioned such that the plunger surface 222 engages the plunger adjuster 118, for example the distal threaded body end 173 of the plunger adjuster 118. The position of the plunger adjuster 118 relative to the plunger 218 may be adjusted by rotating the enlarged proximal head 170 to rotate the threaded body 172 within the plunger adjuster opening 116 to extend the distal threaded body end 173 into the chassis cavity 157. The plunger adjuster 118 may be repositioned for example to engage the plunger surface 222 and/or to prime the fluid (e.g., to remove excess gas) stored in the syringe body lumen. For example, the plunger adjuster 118 may be rotated by 12π to prime the fluid stored in the syringe body lumen. For example, the plunger adjuster 118 may be rotated 2-10 times around (e.g., a 360 degree rotation), for example 6 times, as shown in FIG. 45A for example, to prime the fluid.

To release the fluid stored in the syringe body lumen, the chassis handle 114 may be pulled in a proximal direction to move the chassis assembly 104 relative to the base assembly 102, for example, as shown in FIGS. 45B-C. In some embodiments, the base assembly 102 may be held with the other hand to stabilize the base assembly 102 as the chassis assembly 104 is moved. In some embodiments, the base assembly 102 may stay in place without user involvement, for example, the base assembly 102 may be weighted to remain fixed.

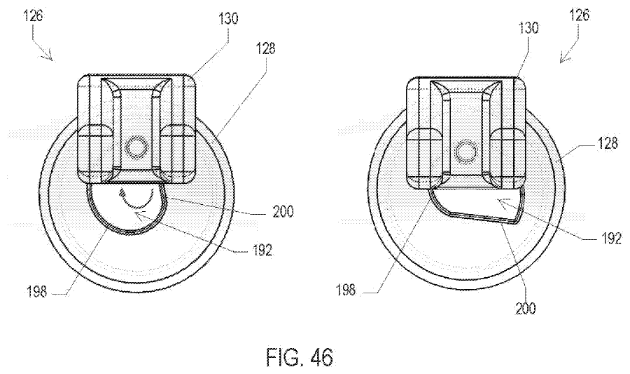

As the chassis assembly 104 is moved in a linear direction, the cam assembly 126 moves with the chassis assembly 104 in the linear direction. As the cam assembly 126 is linearly displaced, the gear 136 rotates as the plurality of gear teeth 212 engage the plurality of gear rack teeth 152. The rotation of the gear 136 rotates the cam 128. For example, the cam 128 may rotate between 90 degrees to 300 degrees, for example 270 degrees. As the cam opening 192 rotates, the contact point between the follower pin 204 and the cam opening 192 edge, e.g., the arcuate edge 198, changes, thereby changing the radius r, e.g., from a larger to a smaller radius, and resulting in linear displacement of the syringe follower 130 in the proximal direction, for example, as shown in FIG. 46. For example, the linear displacement of the cam follower 130 may be the difference between the initial radius and the final radius. For example, if the cam 128 rotates 270 degrees, the initial radius r is 15 and the final radius r is 7.5, the cam follower 130 will be displaced 7.5 mm. The pitch diameter of the gear 136 may be between 45 mm and 60 mm, for example 52.5 mm. If the pitch diameter of the gear 136 is 52.5 mm and the cam 128 rotates 270 degrees, the linear displacement of the cam assembly 126 (and chassis assembly 104) is 123.7 mm, as determined by the following equation:

$$\frac{270}{360} \cdot 52.5 mm \cdot π = 123.7 mm$$

As the prongs 206a,b move in the proximal direction, they push against the flange 220 of the syringe body 216, moving the syringe body 216 relative to the chassis top member 112a and the plunger 218 in the proximal direction, for example, as shown in FIG. 45C. For example, where the cam follower 130 is displaced 7.5 mm, the relative displacement between the syringe body 216 and the plunger 218 is 7.5 mm. The ratio between the displacement of the syringe body 216 relative to the plunger 218 and the total linear displacement of the syringe body 216 (and any component fixedly coupled thereto, e.g., a needle or catheter) is equal to:

$$\frac{7.5}{131.2} ≅ 0.05$$

The total displacement of the syringe body 216 (and any component fixedly coupled thereto, e.g., a needle or catheter) is equal to the sum of the distance the chassis assembly 104 is retracted and the displacement of the syringe body 216 relative to the chassis assembly 104. In the example where the chassis assembly 104 is moved 123.7 mm and the syringe body 216 is displaced 7.5 mm relative to the chassis assembly 104, the total displacement of the syringe body 216 (and any component fixedly coupled thereto) is 131.2 mm. The plunger 218 remains in place due to engagement with the plunger adjuster 118 as the syringe body 216 is moved. As the syringe body 216 is moved relative to the plunger 218, fluid stored within the lumen presses against the plunger 218 and is pushed out the dispense tip. The amount of fluid released is proportional to the distance the syringe body 216 is displaced, which is further proportional to the distance the chassis assembly 104 is moved relative to the base assembly 102. The amount of fluid to be released depends on the volume taken up by the needle or introducer (e.g., catheter, cannula, etc.) being retracted and forming the tissue tract, which depends on the size (e.g., diameter and length) of the needle or introducer. For example, a needle or introducer with an outer diameter of 1.6 mm and having a length 100 mm, occupies a volume of 200 mm³, as determined by the following equation:

$$100 mm \cdot {( \frac{1.6}{2} mm )}^{2} π ≅ 200 {mm}^{3}$$

As such, the volume that needs to be filled by sealant is 200 mm³ (0.2 ml). Because the amount of sealant released is proportional to the movement of the syringe body 216 relative to the plunger 218, in this example, the syringe body 216 needs to be moved 4-5 mm relative to the plunger 218 to inject 200 mm³ of sealant. The injection control device 100 described herein, ensures this precise movement of the syringe body 216 relative to the plunger 218 to provide the exact or near exact amount of fluid/sealant required to properly fill the tissue tract.

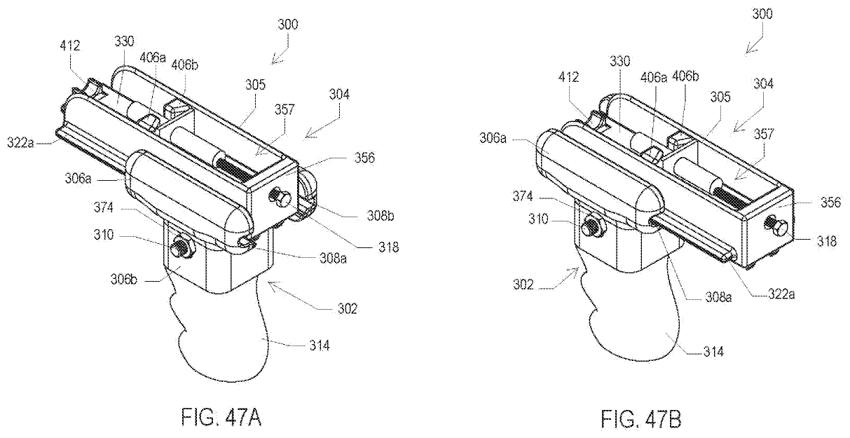

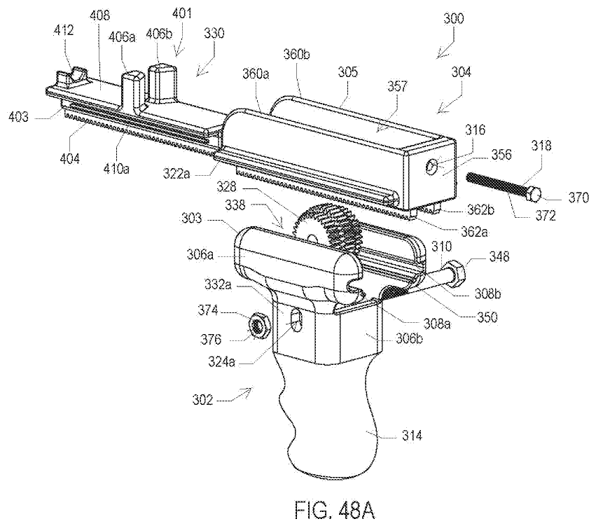

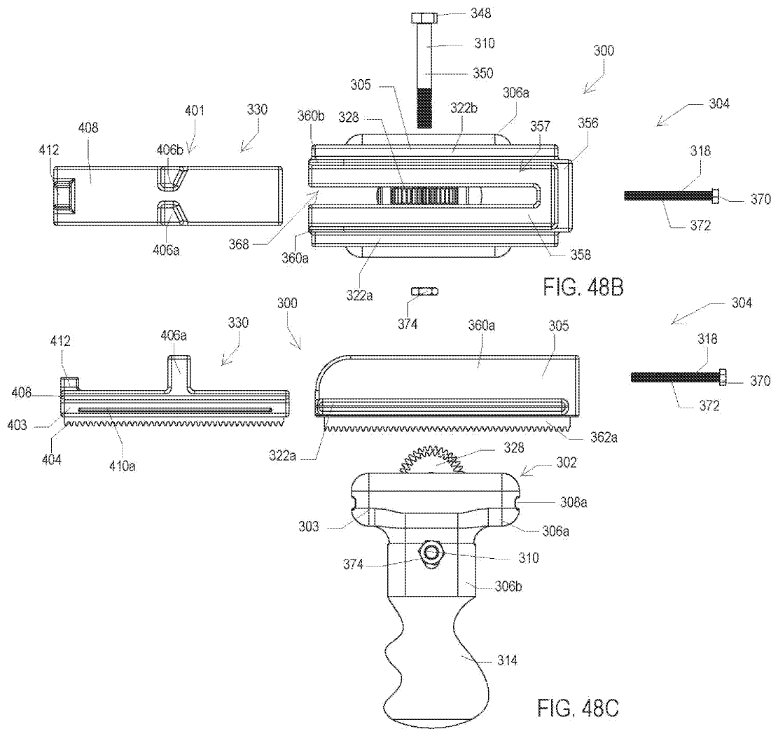

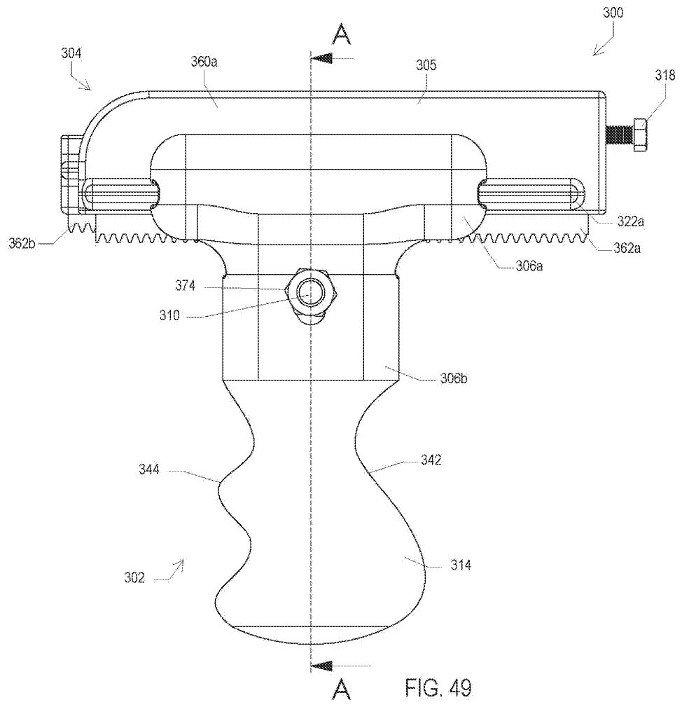

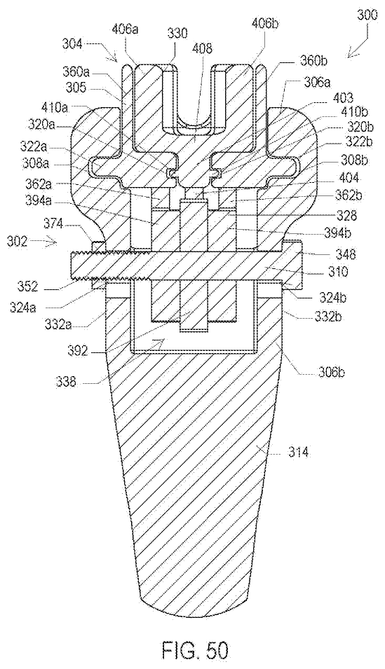

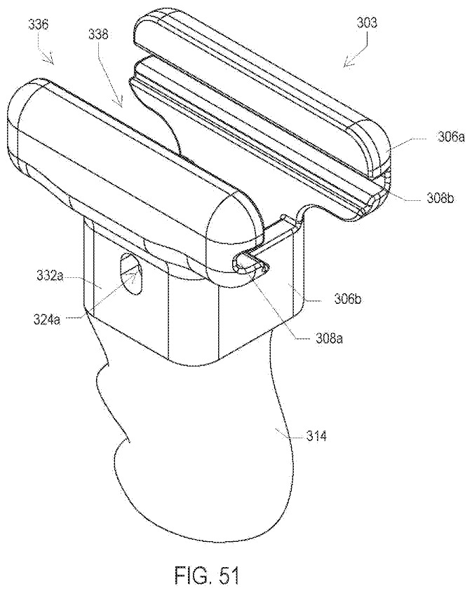

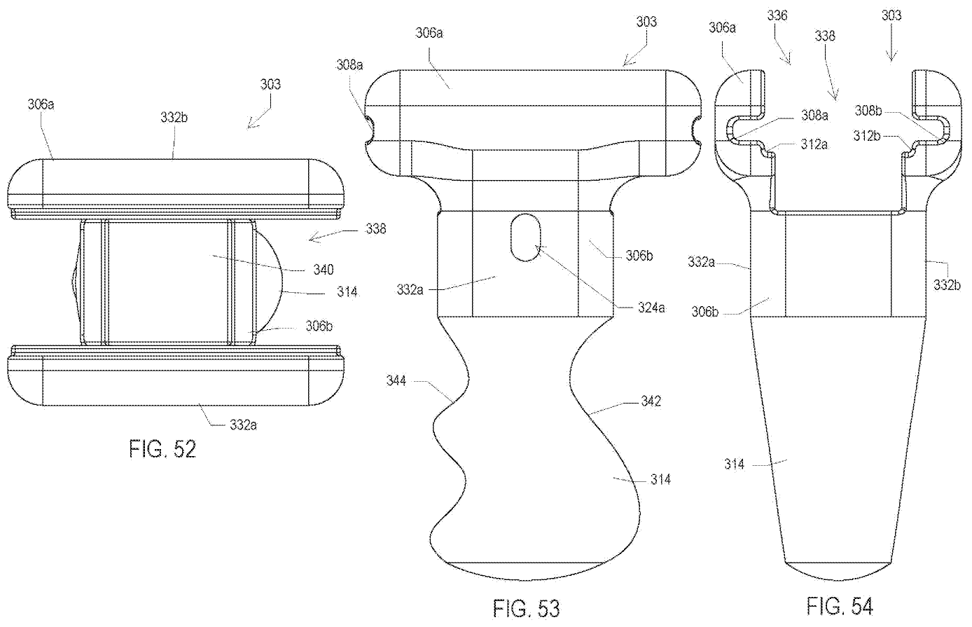

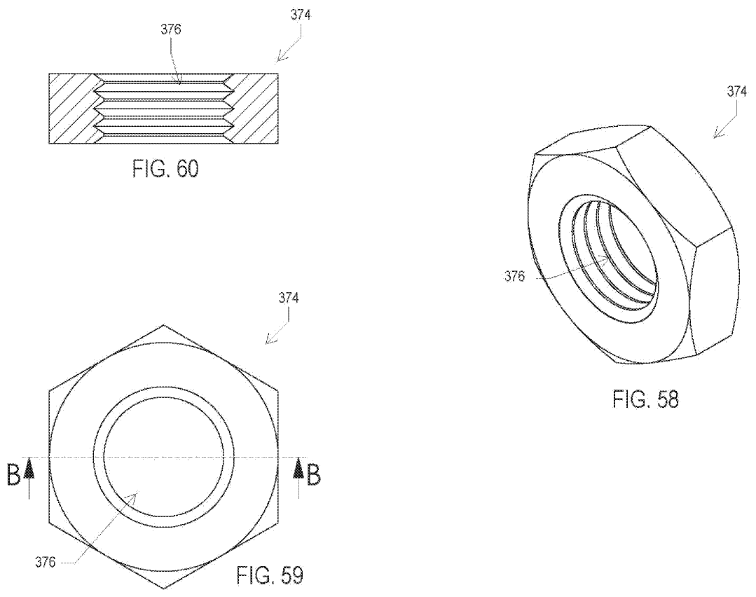

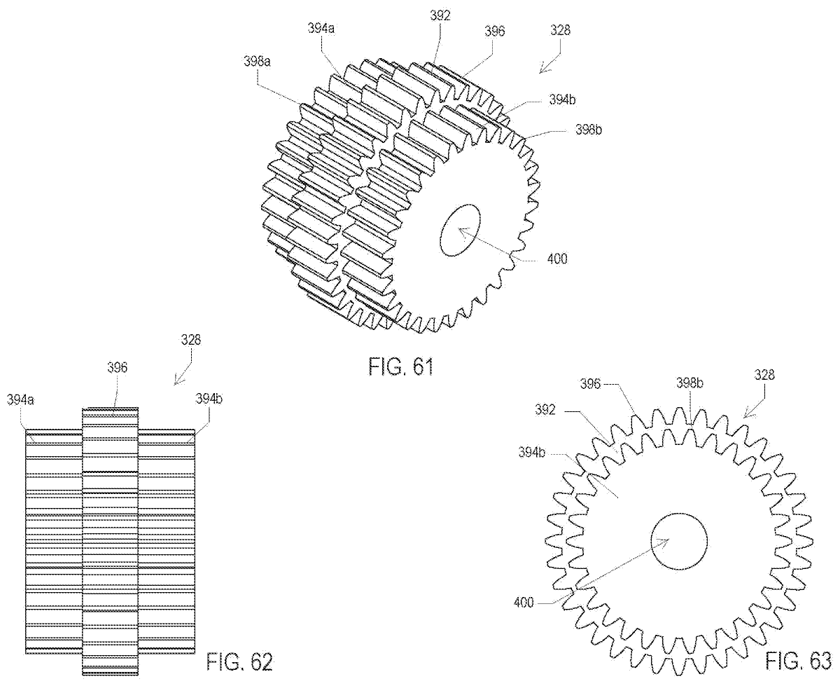

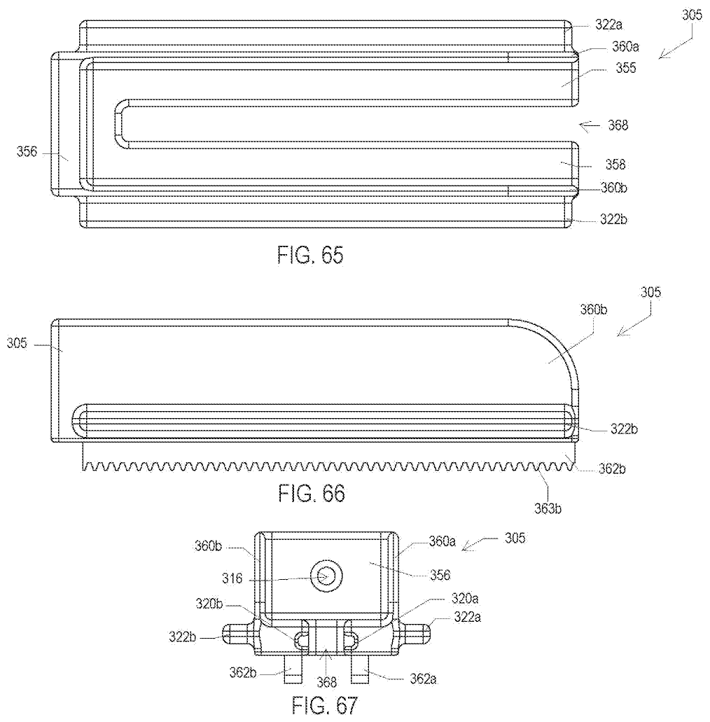

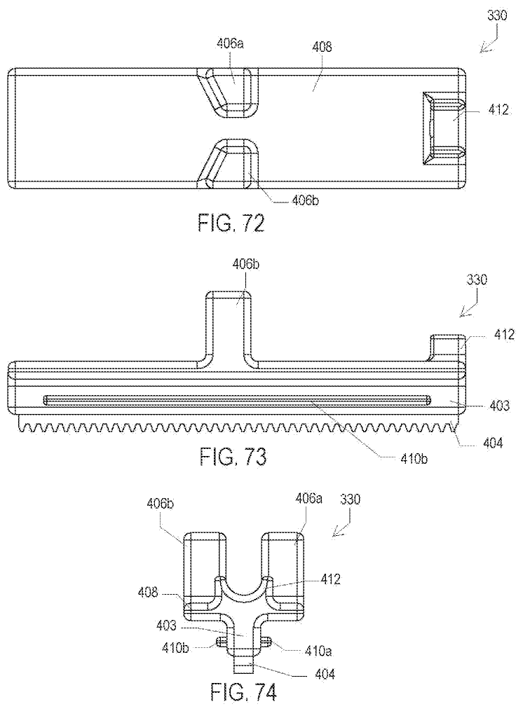

With reference to FIGS. 47A-74B, an injection or delivery control device 300 is disclosed in accordance with another embodiment. FIGS. 47A-B show perspective views of the injection control device 300 in extended and retracted configurations, as discussed in more detail below. FIG. 48A is a perspective exploded view of the injection control device 300 of FIGS. 47A-B, FIG. 48B is a top plan exploded view of the injection control device 300 of FIGS. 47A-B, and FIG. 48C is a left side exploded view of the injection control device 300 of FIGS. 47A-B. FIG. 49 is a left side view of the injection control device 300 of FIGS. 47A-B. FIG. 50 is a cross section view of the injection control device 300 of FIGS. 47A-B taken along line A-A of FIG. 49. As shown in FIGS. 47A-50, the injection control device 300 may include a base assembly or member 302 and a chassis assembly or member 304. The base assembly 302 may include an upper housing member 306a and a lower housing member 306b, two opposing base slots 308a,b, a gear axle 310, a co-centric gear 328, and a handle 314. The chassis assembly 304 may include a chassis body 305, a plunger adjuster 318, and a syringe body holder 330. As discussed in more detail below, the syringe body holder 330 is slidably engaged with the chassis assembly 304 by corresponding rails and slots, and the chassis assembly 304 is slidably engaged with the base assembly 302 by corresponding rails and slots, with both the syringe body holder 330 and chassis assembly 304 movably engaged to the co-centric gear 328.

With reference to FIGS. 48A-70, the base assembly 302 will now be discussed in more detail. As shown, the base assembly 302 includes a base housing 303, a gear axle 310, and a co-centric gear 328. The base housing 303 may include an upper housing member 306a and a lower housing member 306b. As shown, the upper and lower housing members 306a,b are an integral component; however, it is contemplated that the upper and lower housing members 306a,b may be separate components fixedly coupled together. As shown, the lower housing member 306b has a rectangular shape with rounded corners; however, other shapes are contemplated, for example, one or more sides may be rounded. The lower housing member 306b includes first and second opposing base sidewalls 332a,b. The upper housing member 306a forms an upper extension of the first and second opposing base sidewalls 332a,b. The first and second opposing base sidewalls 332a,b curve outwardly (in a direction away from one another) to form the upper housing member 306a. The opposing base sidewalls 332a,b are separated by an interior opening 336 to a base cavity 338 formed by the opposing base sidewalls 332a,b and an inner bottom wall 340. Two opposing base slots 308a,b are defined within an inner surface of the first and second opposing base sidewalls 332a,b, respectively, of the upper housing member 306a. While the opposing base slots 308a,b are depicted as recessed within the opposing base sidewalls 332a,b, it is also contemplated that the opposing base slots 308a,b may be rails extending from the opposing base sidewalls 332a,b. As shown in FIG. 54, the opposing base slots 308a,b may be stepped with respective slot steps 312a,b. For example, as shown, the interior opening 336 may be larger between the opposing base sidewalls 332a,b above the opposing base slots 308a,b than the size of the interior opening 336 below the opposing base slots 308a,b, and the slot steps 312a,b account for this difference.

The first and second opposing base sidewalls 332a,b of the lower housing member 306b may define a first and second axle aperture 324a,b, respectively. While neither the first nor second axle apertures 324a,b include threading, it is contemplated that one or both of the first and second axle apertures 324a,b may be threaded. The first and second axle apertures 324a,b are positioned on the respective first and second opposing base sidewalls 332a,b to share the same central axis.

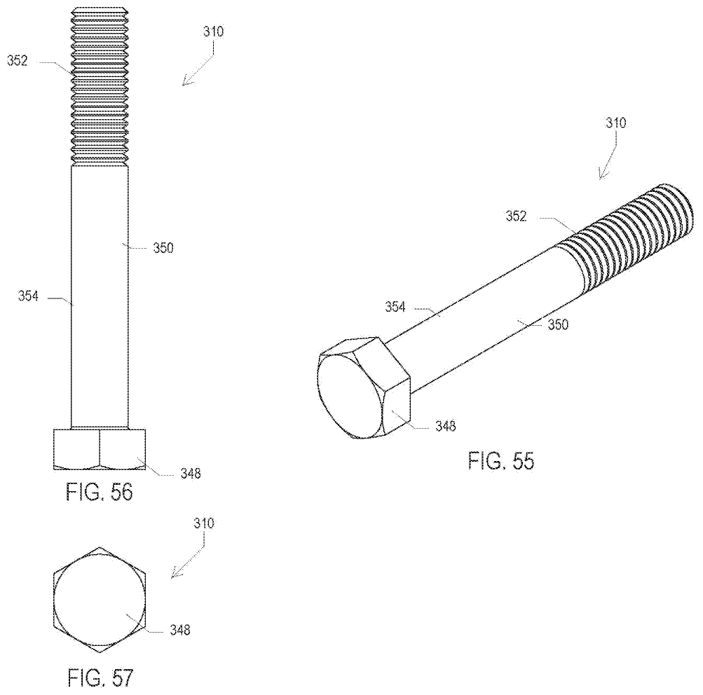

A gear axle 310 is disposed within the first and second axle apertures 324a,b, extending through the base cavity 338. As shown in FIGS. 55-57, the gear axle 310 may include a gear axle head 348 having a gear axle body 350 extending therefrom. The gear axle head 348 may have a larger circumferential dimension compared to the gear axle body 350. As shown, the gear axle head 348 has a hexagonal shape; however, other shapes are contemplated, for example, a circular shape. As one example, the gear axle 310 may be a bolt, screw, or other similar fastener. The gear axle body 350 may have a smooth surface 354 at a proximal end (e.g., the end near the gear axle head 348) and a threaded surface 352 at a distal end; however, it is contemplated that the gear axle body 350 may have a single texture, such as threaded or smooth. As shown in FIG. 50, the gear axle 310 may be positioned within the base housing 303 such that the threaded surface 352 extends through the first axle aperture 324a and engages the threading of the securing mechanism 374, the smooth surface 354 extends through the second axle aperture 324b, the gear axle head 348 engages an outer surface of the second base sidewall 332b, and at least part of the gear axle body 350 extends across the base cavity 338. However, it is contemplated that the orientation of the gear axle 310 may be reversed such that the smooth surface 354 extends through the first axle aperture 324a, the gear axle head 348 engages an outer surface of the first base sidewall 332a, and the threaded surface 352 extends through the second axle aperture 324b to engage with the threading of the securing mechanism 374.

The gear axle 310 may be fixedly coupled to the base housing 303 by a securing mechanism 374. As shown in FIGS. 58-60, the securing mechanism 374 may include a threaded aperture 376 for receiving the threaded surface 352. The depicted securing mechanism 374 has a hexagonal shape. As one example, the securing mechanism 374 may be a nut or other similar fastener. The securing mechanism 374 may be rotatably engaged to the threaded surface 352 of the gear axle body 350 to prevent the gear axle 310 from moving. As shown in FIGS. 49-50, when the securing mechanism 374 is secured, the securing mechanism may engage an outer surface of the first base sidewall 332a; however, if the gear axle 310 is in the opposite configuration, the securing mechanism 374 may engage an outer surface of the second base sidewall 332b.

A co-centric gear 328 is rotatably coupled to the gear axle 310. As shown in FIGS. 61-63, the co-centric gear 328 may include a large gear member or central gear 392, and a small gear member or lateral gears 394a,b. The central gear 392 includes a plurality of central gear teeth 396. The lateral gears 394a,b include a plurality of lateral gear teeth 398a,b, respectively. As shown, the central gear 392 has a greater pitch diameter than the pitch diameter of the lateral gears 394a,b. For example, the central gear 392 may have a pitch diameter between 22 mm and 42 mm, for example, 36 mm. As another example, the lateral gears 394a,b may have a pitch diameter between 20 mm and 40 mm, for example 34 mm. The central gear 392 and lateral gears 394 are fixedly coupled together and share an axis of rotation, such that the central gear 392 and lateral gears 394 have the same angular velocity when the co-centric gear 328 is rotated. A gear aperture 400 may be defined within a center of the co-centric gear and defines the axis of rotation of the central gear 392 and lateral gears 394. The gear aperture 400 may receive the gear axle 310 such that the co-centric gear 328 is rotatably coupled to the gear axle 310. In other words, the co-centric gear 328 axis of rotation is aligned with the axis defined by the gear axle 310. As shown in FIG. 50, the co-centric gear 328 may be positioned relative to the gear axle 310 such that the smooth surface 354 extends through the gear aperture 400, for example to facilitate rotation of the co-centric gear 328 relative to the gear axle 310. The co-centric gear 328 is held in a vertical position by the gear axle 310 within the base cavity 338. For example, the co-centric gear 328 may be positioned in a central location in the base cavity 338, for example, equal distance from either base sidewall 332a,b.

In several embodiments, a handle 314 may be included with the base assembly 302. For example, as shown in FIGS. 47A-48A, 48C-51, and 53-54, the handle 314 is coupled to the lower base member 306b. In the depicted example, the handle 314 extends below the lower base member 306b, in a vertical orientation; however, it is also contemplated that the handle may extend in a horizontal orientation. As shown, the handle 314 has an ergonomic shape with a proximal handle surface 342 and a distal handle surface 344. The proximal handle surface 342 has a concave upper portion and convex lower portion, for example, for a user's palm to easily grasp. The distal handle surface 344 has two parallel indentations or concave areas, for example, for a user's fingers to easily grasp. Other ergonomic shapes are contemplated, for example, the handle surfaces may be substantially flat or the handle 314 may have a ring or loop shape.

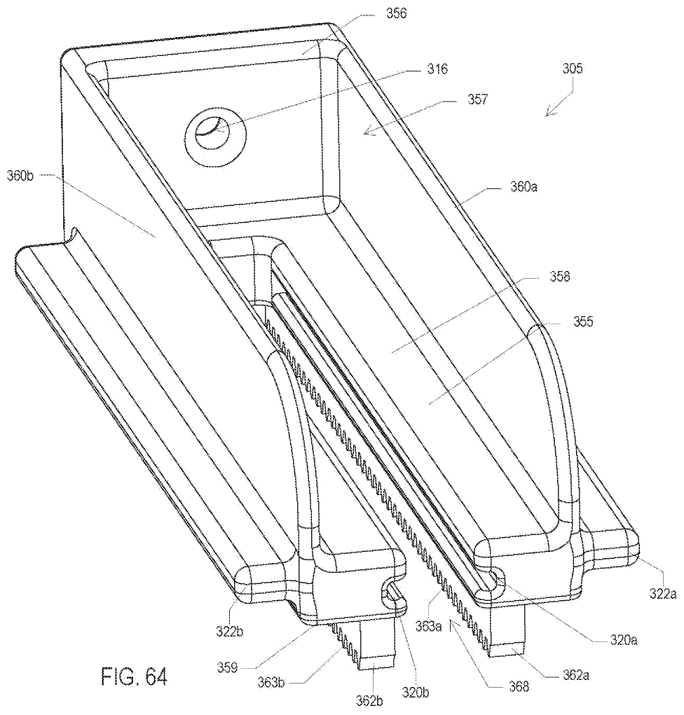

With reference to FIGS. 64-70, the chassis assembly 304 will now be discussed in more detail. The chassis assembly 304 may include a chassis body 305, a plunger adjuster 318, and a syringe body holder 330. The chassis body 305 may include a proximal wall 356, opposing first and second chassis sidewalls 360a,b, and a chassis bottom wall 358. The proximal wall 356, opposing first and second chassis sidewalls 360a,b, and chassis bottom wall 358 may define a chassis cavity 357. The chassis bottom wall 358 may define a chassis top surface 355 and a chassis bottom surface 359. The chassis bottom wall 358 may have a chassis opening 368 defined therethrough. The chassis opening 368 may be further defined by opposing chassis slots 320a,b perpendicularly oriented relative to the chassis top surface 355. However, it is contemplated that the opposing chassis slots 320a,b may be rails extending into the chassis opening 368.

The chassis opening 368 may be further defined by parallel chassis gear racks 362a,b extending from the chassis bottom surface 359. As shown, the chassis gear racks 362a,b are positioned on either side of the chassis opening 368. The chassis gear racks 362a,b comprise a plurality of chassis gear teeth 363a,b, respectively. As shown in FIG. 64, the chassis gear racks 362a,b extend along substantially the entire length of the opposing chassis slots 320a,b.

The first and second chassis sidewalls 360a,b may have opposing chassis rails 322a,b, respectively, extending therefrom. For example, as shown in FIG. 64, the opposing chassis rails 322a,b extend in opposing outward directions (e.g., outside the chassis cavity 357). As shown, the opposing chassis rails 322a,b are positioned on a lower portion of the opposing first and second chassis sidewalls 360a,b, e.g., in a vertical position proximate the chassis bottom wall 358. However, it is contemplated that the vertical positioning of the opposing chassis rails 322a,b on the opposing first and second chassis sidewalls 360a,b may vary. The sizing and shape of the opposing chassis rails 322a,b is selected to correspond with the sizing and shape of the corresponding opposing base slots 308a,b. It is also contemplated that the opposing chassis rails may be slots defined within the first and second chassis sidewalls 360a,b, for example, where the base assembly 302 includes opposing base rails, instead of opposing base slots 308a,b, extending from the opposing base sidewalls 332a,b.

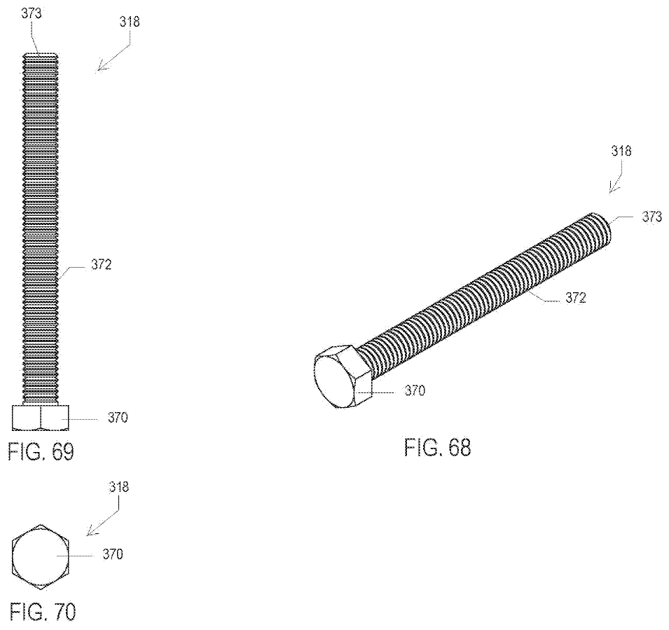

The proximal wall 356 may define a plunger adjuster opening 316 therethrough. The plunger adjuster opening 316 may including threading, for example, to receive a threaded portion of a plunger adjuster, for example, plunger adjuster 318 shown in FIGS. 68-70. As shown, the plunger adjuster 318 has an enlarged proximal head 370 with a threaded body 372 having a distal threaded body end 373. As shown, the enlarged proximal head 370 has a hexagonal shape; however, other shapes are contemplated, for example, a circular shape. As one example, the plunger adjuster 318 may be a bolt, screw, or other similar fastener. In some embodiments, the plunger adjuster 318 may include a securing mechanism, for example securing mechanism 374 depicted in FIGS. 58-60. The securing mechanism 374 may be rotatably engaged to the threaded body 372 of the plunger adjuster 318 on the distal side of the proximal wall 356 to prevent the plunger adjuster 318 from moving. However, in the depicted embodiment, the securing mechanism 374 is omitted.

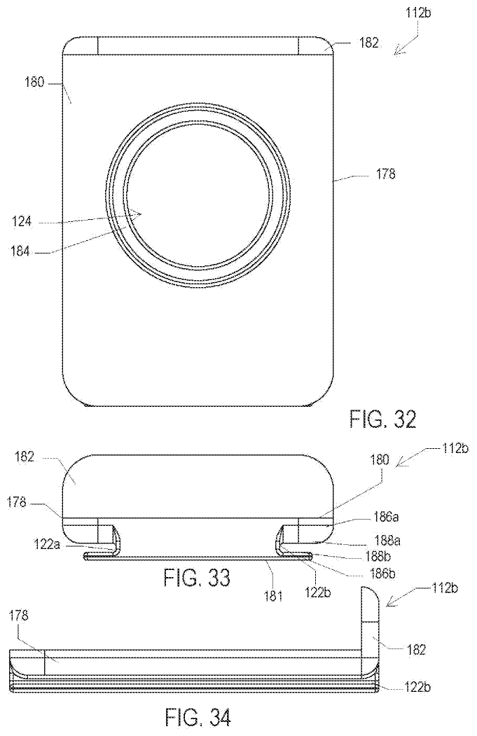

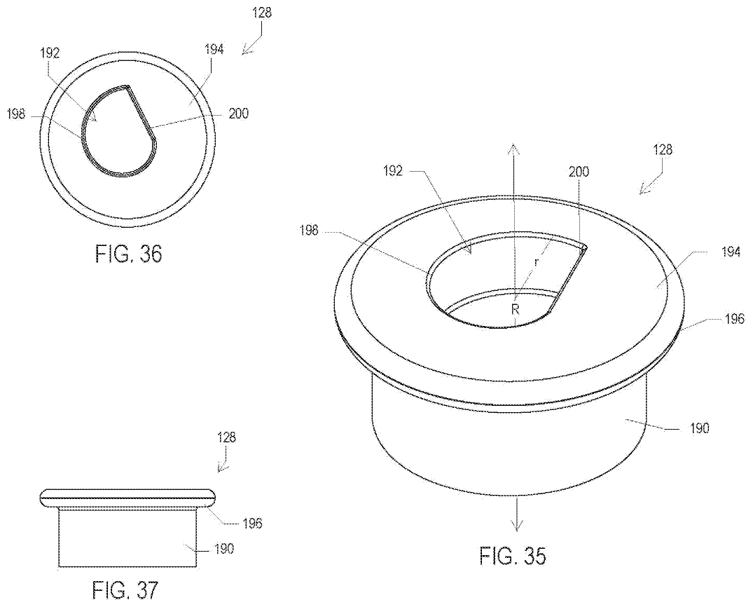

In some embodiments, the proximal wall 356 may further include a handle coupled on a proximal side of the chassis body 305. For example, the handle may be similar to the handle 114 depicted in the embodiment shown in FIGS. 1-46 to facilitate movement of the chassis assembly 304 relative to the base assembly 302 by a user.

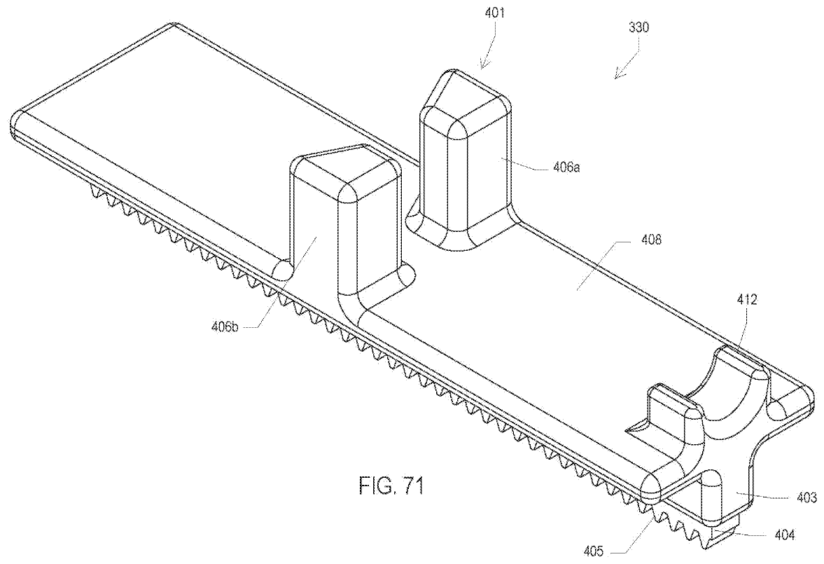

The syringe body holder 330 may be positioned within the chassis opening 368 and slidably engaged with the chassis body 305. As shown in FIGS. 71-74, the syringe body holder 330 may include a holder base 408 having a syringe body engagement member 401 for engaging a syringe body. For example, as shown, the syringe body engagement member 401 may include two parallel prongs 406a,b extending from the holder base 408; however, other structures are contemplated to engage and secure a syringe body, for example, a ridge, lip or hook extending from the holder base 408, or an opening to latch a part of the syringe body. The syringe body holder 330 may further include a syringe extension holder 412 at a distal end of the holder base 408. As shown, the syringe extension holder 412 extends from the holder base 408 and forms a concave, U-shape for receiving an extension of the syringe, for example, a longer syringe body or a needle or other structure coupled thereto. As shown, the syringe extension holder 412 has a smaller height (e.g., extends a shorter distance from the holder base 408) than the syringe body engagement member 401.

The syringe body holder 330 may further include a gear rack extension 403 extending from the holder base 408 in a downward direction opposite the prongs 406a,b. The gear rack extension 403 includes a holder gear rack 404 and positions the holder gear rack 404 a distance below the base; however, it is also contemplated that the gear rack extension 403 may be omitted and the holder gear rack 404 may be coupled to a bottom surface of the holder base 408. As shown, the holder gear rack 404 may include a plurality of holder gear rack teeth 405. The holder gear rack 404 is positioned along a central longitudinal axis of the holder base 408. The holder gear rack 404 may be positioned between the chassis gear racks 362a,b. As shown in FIG. 50, the holder gear rack 404 extends a shorter distance below the chassis assembly 304 than the chassis gear racks 362a,b.

The syringe body holder 330 may include opposing holder rails 410a,b extending from the gear rack extension 403 in opposing directions, for example, positioned on opposing sides of the holder gear rack 404, as shown in FIGS. 73-74, for example. The opposing holder rails 410a,b are sized and shaped to correspond to the corresponding chassis slots 320a,b. The opposing holder rails 410a,b may be slidably engaged to the chassis slots 320a,b, respectively. It is contemplated that the opposing holder rails 410a,b may be positioned on opposing lateral sides of the holder base 408, for example where the gear rack extension 403 is omitted. It is further contemplated that the opposing holder rails 410a,b may be slots defined within the holder body 408, for example, where the chassis body 305 includes opposing chassis rails extending into the chassis opening 368, instead of opposing chassis slots 320a,b.

An assembled injection control device 300 will now be discussed in more detail. For example, as shown in FIGS. 47A-50, the chassis assembly 304 may be slidably engaged with the base assembly 302. For example, the chassis assembly 304 may be positioned within the interior opening 336 to the base cavity 338. The opposing chassis rails 322a,b may be slidably engaged with corresponding opposing base slots 308a,b of the base assembly 302.

The co-centric gear 328 may engage the holder gear rack 404 and the chassis gear racks 362a,b. For example, the central gear 392 may engage the holder gear rack 404 and the lateral gears 394a,b may engage the chassis gear racks 362a,b, respectively. For example, the central gear teeth 396 may engage the holder gear rack teeth 405 and the lateral gear teeth 398a,b may engage the chassis gear rack teeth 363a,b, respectively. The difference in pitch diameter between the central gear 392 and the lateral gears 394a,b may correspond to the difference in height between the holder gear rack 404 and the chassis gear racks 362a,b, allowing the gears to engage the respective gear racks.

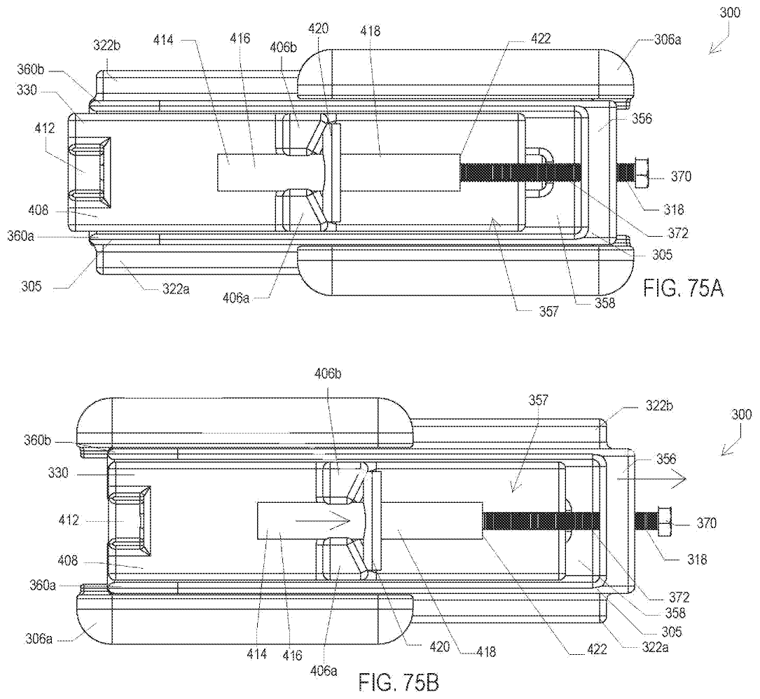

In operation, a delivery device, such as a syringe, may be placed within the chassis cavity 357. For example, FIGS. 75A and 75B show the injection control device 300 being used with an exemplary syringe 414. As shown, the exemplary syringe 414 includes a syringe body 416 defining a lumen and a plunger 418 positioned at least partially within the lumen and movably coupled to the syringe body 416. The syringe body 416 may include a flange 420 at a proximal end of the syringe body 416 and a dispense or delivery tip at a distal end of the syringe body 416. The plunger 418 may include a plunger surface 422 at a proximal end that is shaped to allow a user to press on the plunger 418, for example a flat surface. The syringe body lumen may house a viscous fluid, for example, a sealant. The sealant may be any biocompatible material or bio glue used to seal lesions or excision sites in a patient. The plunger 418 is configured to press against the fluid stored within the syringe body lumen when the plunger 418 and syringe body 416 are moved relative to one another to push the fluid out of the delivery tip.

In several embodiments, the syringe plunger 418 may be positioned proximate a proximal end of the chassis body 305, e.g., adjacent the proximal wall 356, and the syringe body 416 may be positioned proximate a distal end of the chassis assembly 304. For example, the syringe body 416 may be positioned between the prongs 406a,b of the syringe body holder 330. The syringe body 416 may be positioned such that the flange 420 at the proximal end is positioned on a proximal side of the prongs 406a,b, for example, engaging a proximal surface of the prongs 406a,b. The plunger 418 may be positioned such that the plunger surface 422 engages the plunger adjuster 318, for example the distal threaded body end 373 of the plunger adjuster 318. The position of the plunger adjuster 318 relative to the plunger 418 may be adjusted by rotating the enlarged proximal head 370 to rotate the threaded body 372 within the plunger adjuster opening 316 to extend the distal threaded body end 373 into the chassis cavity 357. The plunger adjuster 318 may be repositioned for example to engage the plunger surface 422 and/or to prime the fluid (e.g., to remove excess gas) stored in the syringe body lumen. For example, to prime the fluid, the plunger adjuster 318 may be rotated 2-10 times around (e.g., a 360 degree rotation), for example 6 times, as shown in FIG. 75A for example.

To release the fluid stored in the syringe body lumen, the base handle 314 may be held as the chassis body 305 is pulled in a proximal direction to move the chassis assembly 304 relative to the base assembly 302, for example, as shown in FIG. 75B. In embodiments including a handle on the proximal wall 356 of the chassis body 305, the handle may be pulled to displace the chassis assembly 304 in the proximal direction.

As the chassis assembly 304 is moved in the proximal direction relative to the base, the chassis gear racks 362a,b and the holder gear rack 404 are linearly displaced in the proximal direction, such that the holder gear rack teeth 405 and the chassis gear rack teeth 363a,b engage the respective central and lateral gear teeth 396, 398a,b to rotate the co-centric gear 328 about the gear axle 310 and the axis of rotation. As the central gear 392 rotates and the central gear teeth 396 engage the holder gear rack teeth 405, the central gear 392 moves the holder gear rack 404 at a faster speed than the speed of movement of chassis gear racks 362a,b based on the larger pitch diameter of the central gear 392 compared to the smaller pitch diameter of the lateral gears 394a,b. The faster holder gear rack 404 moves the syringe body holder 330 relative to the chassis body 305 and in the proximal direction. In other words, the syringe body holder 330 moves toward the proximal wall 356 of the chassis body 305. As the prongs 406a,b of the syringe body holder 330 move in the proximal direction, they push against the flange 420 of the syringe body 416, moving the syringe body 416 relative to the chassis body and the plunger 418 in the proximal direction, for example, as shown in FIG. 75B. The plunger 418 remains in place as the syringe body 416 is moved due to engagement of the plunger surface 422 with the plunger adjuster 318. As the syringe body 416 is moved relative to the plunger 418, fluid stored within the lumen presses against the plunger 418 and is pushed out the dispense tip. The amount of fluid released is proportional to the distance the syringe body 416 is displaced, which is further proportional to the distance the chassis assembly 304 is moved relative to the base assembly 302.

The total linear displacement of the syringe body 416 is proportional to the angle rotation of the co-centric gear 328. For example, to move the syringe body 416 (and any component fixedly coupled thereto, e.g., a needle) a total linear distance of 100 mm, the total angle rotation of the gears will be 5.55 (radian), as determined by the following equation:

$$\frac{100 mm}{( \frac{36 mm}{2} )} = 5.55 ( radian )$$

The total linear displacement of the chassis body 305 (e.g., the plunger 418) is also proportional to the angle rotation of the co-centric gear 328. In this example, the total displacement of the chassis body 305 (and plunger 418) is 94.44 mm, as determined by the following equation:

$$5.55 radian \times \frac{34 mm}{2} = 94.44 mm$$

The relative displacement of the syringe body 416 relative to the plunger 418 can be determined by the difference between the total linear displacement of the syringe body 416 and the linear displacement of the plunger 418. In this example, the relative displacement of the syringe body 416 relative to the plunger 418 is 5.56 mm, as determined by the following equation:

100 mm−94.44 mm=5.56 mm

This relative displacement corresponds to the injection of 0.4 ml of glue for an exemplary syringe with two chambers having an internal diameter of 4.4 mm and 9.3 mm, respectively. The volume ejected by the device will depend on the gearing differential, and the radius of the syringe used. For the example above, the volume ejected over 100 mm of syringe withdrawal is:

$$V = 5.56 mm \times π \times ( \frac{{4.4}^{2} + {9.3}^{2}}{4} ) = 462 {mm}^{3}$$

The gearing differential and the syringe diameter may be selected to achieve the desired amount of syringe body movement and injection volume. For example, the volume of injectate may be calculated based on the following equation:

$$V = ( \frac{4 . {4}^{2} + 9 . {3}^{2}}{4} ) \cdot π \cdot \frac{{R}_{1} - {R}_{2}}{{R}_{1}} [ \frac{ml}{mm} ] = 0 \text{.083} \cdot ( 1 - \frac{{R}_{2}}{{R}_{1}} ) [ \frac{ml}{mm} ]$$

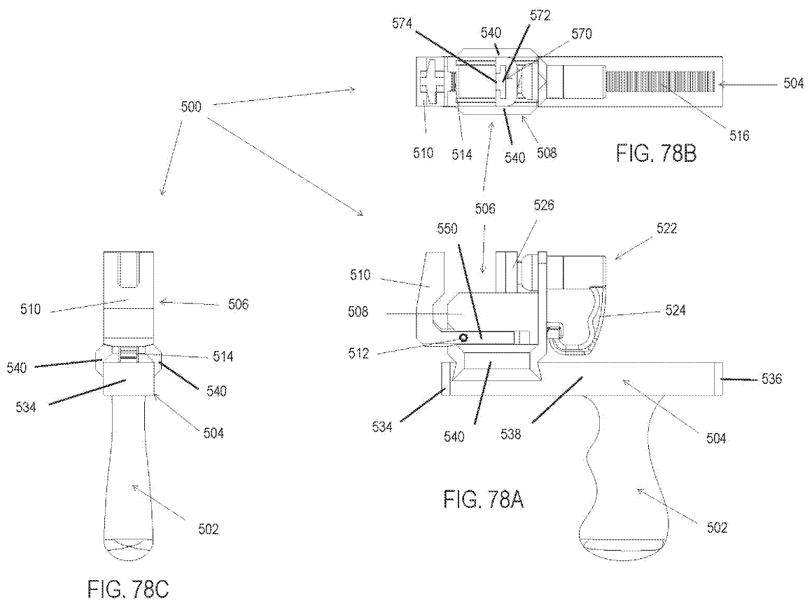

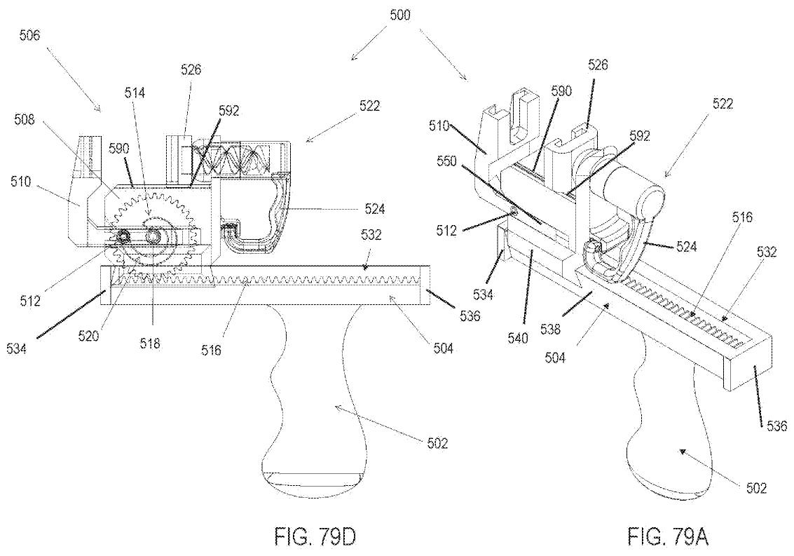

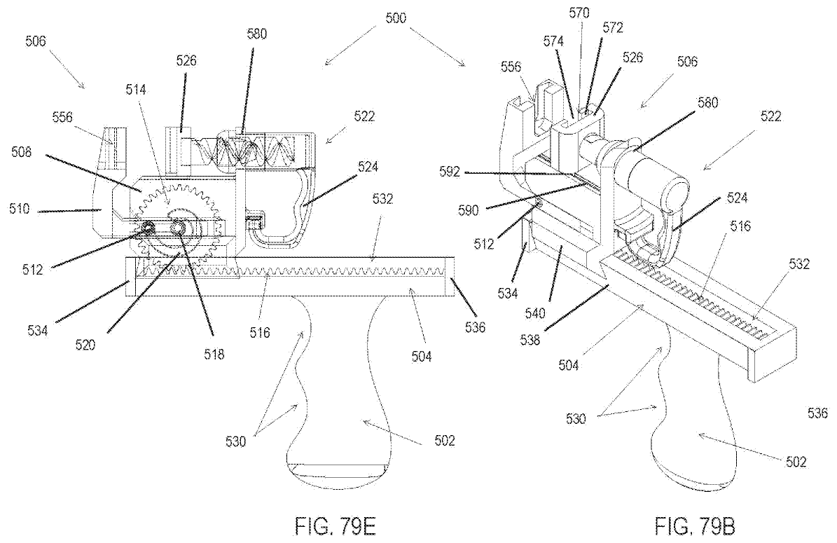

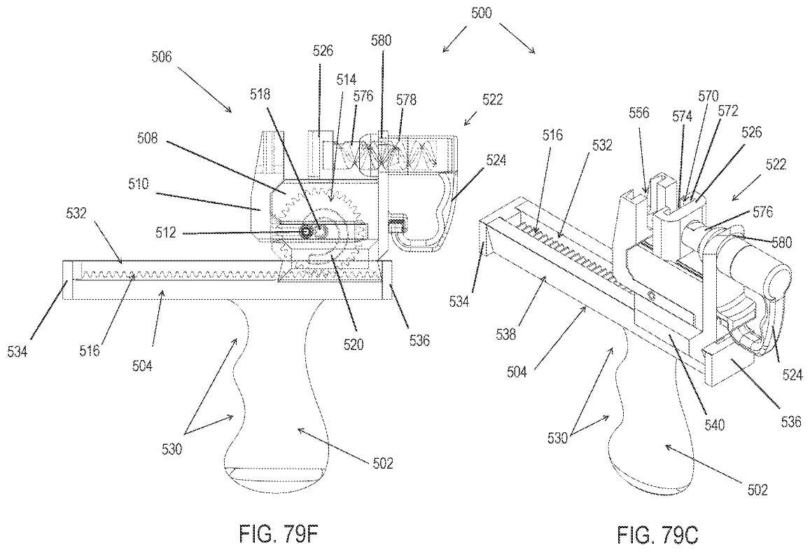

FIGS. 78A to 78C depict another embodiment of an injection control device 500. In this embodiment, the device 500 comprises a primary handle 502 with an elongate body 504 on which the movable chassis or carriage 506 is slidably attached. The chassis 506 comprises a rear chassis 508 that is slidably coupled to the elongate body 504, and a front chassis 510 that is slidably coupled to the rear chassis 508, via a cam follower pin 512 located in a spiral cam gear 514 attached to the rear chassis 508. The spiral cam gear 514 interfaces with a rack 516 located on the elongate body 504. The front chassis 510 may be coupled to the body of a syringe while the rear chassis 508 is coupled to the plunger of the syringe. As the rear chassis 508 is displaced proximally, both the syringe and the syringe plunger are also displaced proximally. However, as depicted in FIGS. 79B, 79C, 79E and 79F, the spiral cam gear 514 rotates along the rack 516, the front chassis 510 is pulled closer to the rear chassis 506 as the follower pin 512 is pulled closer to the axle 518 by the spiral recess 520 of the cam gear 514 and rotation axis of the spiral cam gear 514, which pulls the front chassis 510 closer to the rear chassis 508, which results in a relative displacement between the syringe body and the syringe plunger, to thereby inject the syringe contents as the syringe is withdrawn.

The rear chassis 508 may also comprise a plunger adjustment assembly 522, which may be used to modify the initial plunger position and/or to slightly depress the plunger to prime the syringe for injection, e.g. to evacuate any air bubbles or fill the deadspace in the syringe and/or attached needle or cannula prior to use. The plunger adjustment assembly 522 may comprise a rotatable handle 524 that is threadably coupled to a plunger attachment head 526 that is configured to engage the plunger. As depicted in FIGS. 79A, 79B, 79D and 79E, rotation of the handle 524 results in distal displacement of the plunger attachment head 526, to thereby depress or adjust the plunger position relative to the syringe body that is attached. This adjustment of the plunger position is independent of the change in configuration between the rear chassis 508 and front chassis 510 that occurs with displacement of the rear chassis 508. Further embodiments and details of the injection control device 500 are described below.

As depicted in FIGS. 78A to 80B, the primary handle 502 of the injection control device 500 may be generally orthogonal to the elongate body 504, but in other variations, the angle between the handle 502 and the elongate body 504 may be in the range of 45 to 135 degrees, or 70 to 110 degrees. The handle 502 may comprise generally curved surfaces with one or more smooth finger indentations 530 to facilitate stable gripping. The rack 516 of the elongate body 504 may be located in a trough, recess or cavity 532. The cavity 532 may reduce the overall height of the device 500, and/or reduce the risk of separation of the chassis 506 from the device 500. The rack 516 may have a length in the range of 100 to 300 mm, 120 to 200 mm, or 140 to 150 mm, for example, and the number of teeth may be in the range of 20 to 80, 30 to 60 or 30 to 50, for example. The elongate body 504 may also include an endcap 534 and/or stop flange or stop structure 536. The endcap 534 may facilitate the attachment of the chassis 506 to the elongate body 504 during manufacture, followed by the attachment of the endcap 534 to the elongate body 504 via adhesive, welding or heat melting, for example, to resist removal of the chassis 506 after attachment. The stop flange structure 536 may be provided at the other end of the elongate body to also stop inadvertent separation of the chassis 506. In other examples, however, a second endcap may be provided instead of the stop flange structure 536. The sidewalls 538 of the elongate body 504 may also be tapered toward the centerline of the elongate body 504, which may be engaged by complementary bracket structures 540 of the rear chassis 508 to secure the chassis 506 to the elongate body 504. As depicted in FIGS. 79A to 79C, the endcap 534 and stop flange structure 536 project laterally to limit distal and proximal displacement of the chassis 506 from the elongate body 504. In other variations, however, a groove with a complementary slide structure in the groove may be provided.

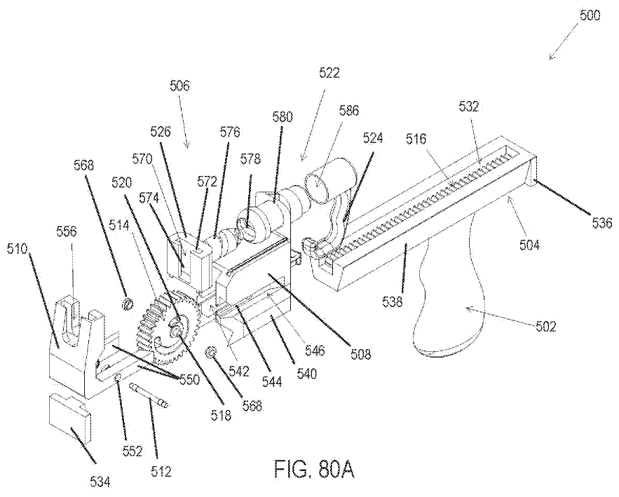

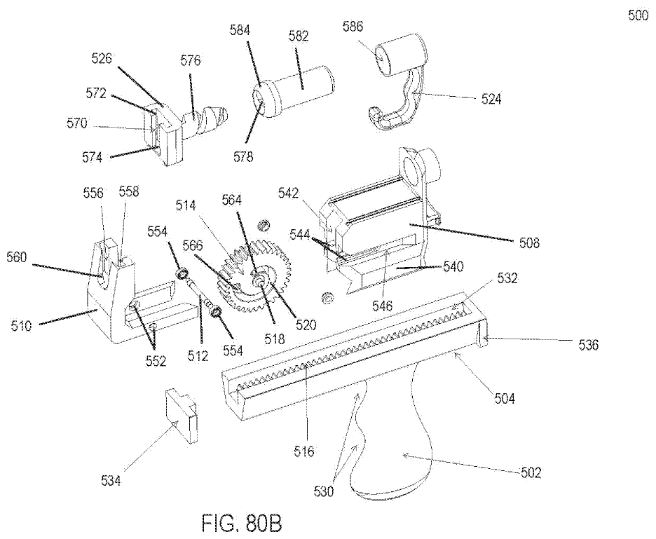

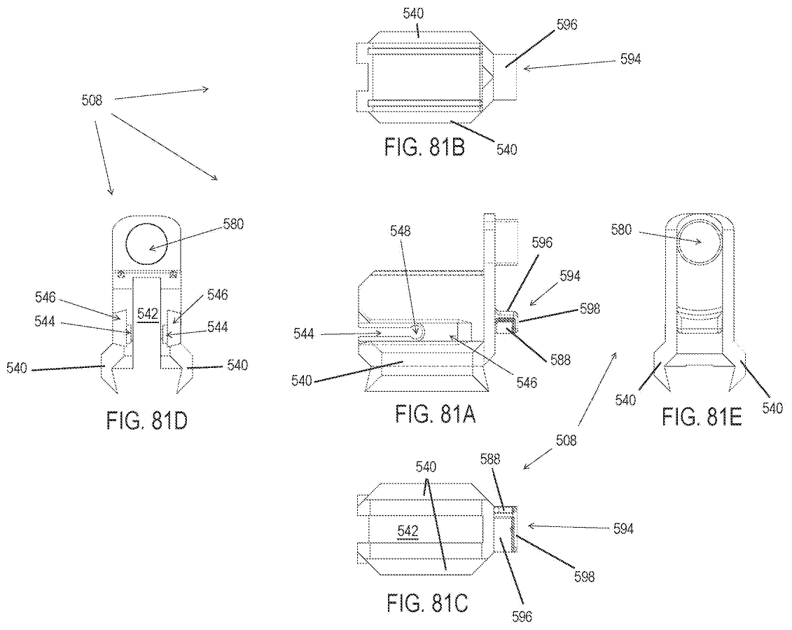

As depicted in FIGS. 80A to 81E, the rear chassis 508 comprises midline slot 542 in which the spiral cam gear resides and bilateral side slots 544 in which the follower pin 512 of the front chassis 510 movably resides. The side slots 544 are located in larger side recesses 546 in which the struts 550 of the front chassis 510 are configured to movably reside. The axle apertures 548 in which the axle 518 of the spiral cam gear 514 are attached may be confluent with the side slots 544 as depicted in FIGS. 80A to 81E, or may be separate in other embodiments.

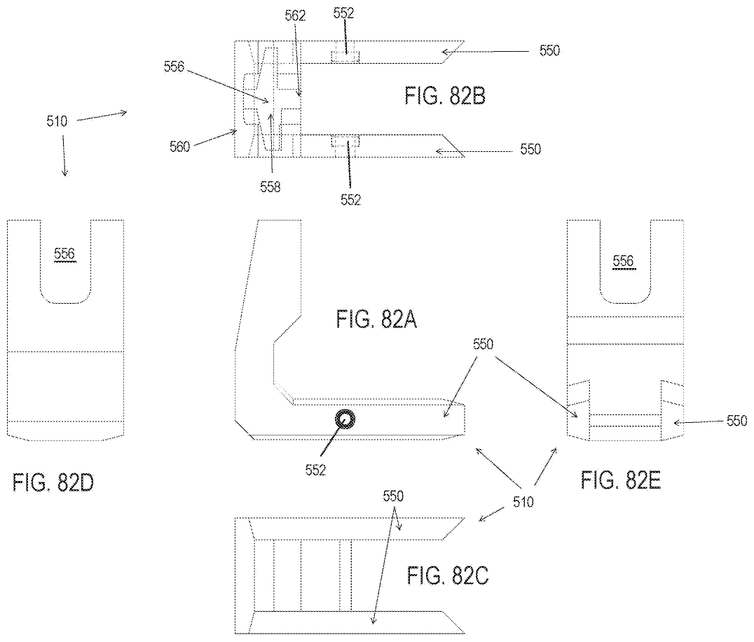

As depicted in FIGS. 80A, 80B and 82A to 82E, the front chassis 510 comprises a general L-shape configuration with two lower elongate struts 550 which are configured to slidably reside in the side recesses 546 of the rear chassis 508. The struts 550 are configured with a longitudinal length that is sufficient to provide the amount of travel configured between the rear and front chasses 508, 510 to provide the desired plunger travel distance. This length may be in the range of 30 to 60 mm, or 35 to 50 mm, for example. The struts 550 each also comprise a follower pin opening 552 which are configured to receive the follower pin 512 of the cam assembly and optionally the securing rings 554 that attach to the ends of the follower pin 512 to secure the pin 512 to the struts 550. The front chassis 510 further comprises a vertical, distal section with a syringe body cavity 556, which is typically configured to receive the syringe body flange located at the proximal end of the syringe body. The syringe body cavity 556 is configured with the top or side opening 558 into which the flange of the syringe body is retained. A distal opening 560 is also provided from which the syringe body may project distally, as well as a proximal opening 562 from which the syringe plunger may project. In other variations, the syringe body cavity 556 may comprise a tension clamp mechanism to hold the syringe body.

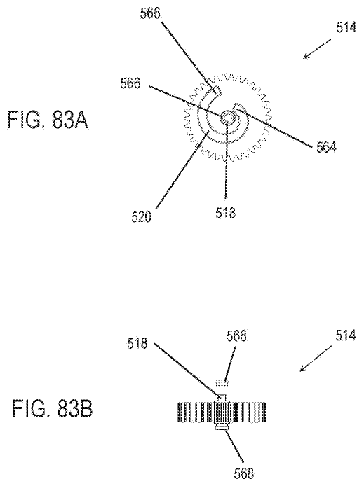

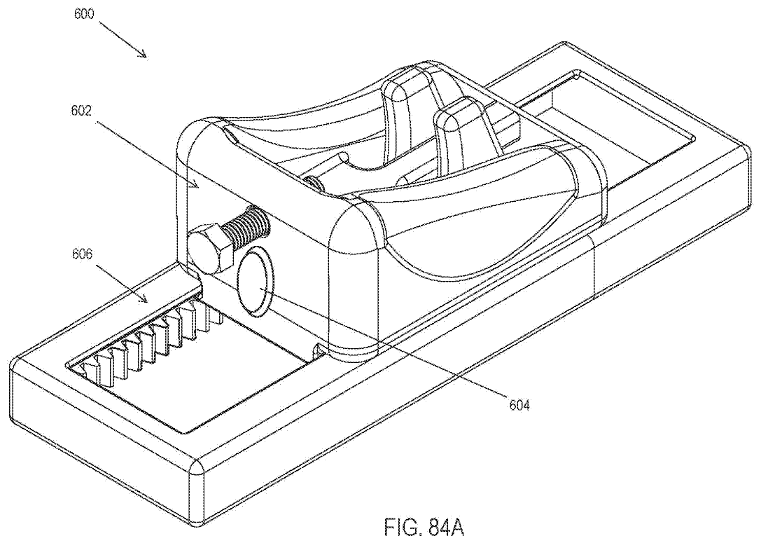

As depicted in FIGS. 80A, 80B, 83A and 83B, the spiral cam gear 514 comprises a spiral recess 520 that has a uniform change in radius across its rotation range, but in other variations, the spiral recess may be configured to a non-uniform change in radius across its rotation range, e.g. provide greater injection of material per unit of withdrawal distance. In this particular embodiment, the spiral recess 520 has a rotational range of slightly less than 360 degrees, but in other variations, the rotational range may be greater or equal to 360 degrees, e.g. 360 to 450 degrees, 360 to 405 degrees, or 360 to 380 degrees, or may be less than 360 degrees, e.g. between 335 degrees and 355 degrees, or 345 to 355 degrees. In this specific example, the spiral recess 520 is configured with a minimum and maximum radius 564, 566 that changes around 8 mm over the rotation range of the gear 514, which is around 100 to 110 mm of linear distance. In other variations, the maximum change in radius may be in the range of 1 to 50 mm, 5 to 20 mm, or 8 to 10 mm, over a linear distance of gear travel in the range of 20 to 250 mm, 50 to 200 mm, 80 to 120 mm, or 90 to 110 mm. The number of teeth per circumference of the spiral cam gear 514 may vary and depend on the configuration of the rack 516, but may be in the range of 15 to 75, 25 to 55 or 25 to 45, for example. The axle 518 of the spiral cam gear 514 may further include an integrated retaining ring and/or separately attachable axle retainer rings 568.

In embodiments comprising the plunger adjustment assembly 522, the plunger cavity 570 is provided on a plunger attachment head 526. The plunger cavity 570 may comprise a side or top opening 572 into which the flange of the plunger is retained. The plunger cavity 570 further comprises a distal opening 574 from which the plunger extends distally toward the syringe body cavity 556. Extending from the plunger attachment head 526 is a helical body 576 which in turn is rotatably received in a complementary helically threaded plunger handle lumen 578. The rotation of the plunger handle 524 results in the longitudinal translation of the plunger attachment head 526 and helical body 576, thereby adjusting the plunger position relative to the syringe body. The plunger adjustment assembly 522 may be located in a flange opening 580 of the rear chassis 508. The handle 524 may be provided in two components that are joined during the manufacturing process, with a distal body 582 containing the helical lumen 578 and comprising a distal flange 584 that limits proximal displacement of the handle 524 from the rear chassis 508. This distal body 582 may be fixedly attached to the handle 524 via adhesive, welding or heat melding to a handle cavity 586. In embodiments of the rear chassis without a plunger adjustment assembly, the plunger engagement or receiving cavity for the plunger flange may be fixedly provided directly on the rear chassis 508.

As the plunger handle 524 is rotated, the helically threaded lumen 578 is rotated, thereby causing longitudinal displacement of the plunger attachment head 526. To resist rotation of the plunger attachment head 526 and deviations from the longitudinal displacement path, the rear chassis 508 and the plunger attachment head 526 may further comprise a sliding interface to constrain the motion of the plunger attachment head 526. This sliding interface may comprise, for example, one or more grooves 590 located on the outer surface of the rear chassis 508, which slidably receive slide structures 592 projecting from the outer surface of the plunger attachment head 526. In this particular example, approximately 3 helical threads are provided over a longitudinal distance of about 50 to 60 mm. In other variations, 1-3 helical threads may be provided over a longitudinal distance in the range of about 20 to 100 mm, 30 to 80 mm, 40 to 70 mm, for example.

As depicted in FIGS. 79A to 79C, the handle 524 may comprise a general J-shaped configuration, with one or more concave finger surfaces to facilitate gripping. To help distribute pulling forces acting on the rear chassis 508 closer to the elongate body 504 of the device 500 during use, the rear chassis 508 may comprise a receiving flange 594 into which the J-shaped end of the handle 524 rotationally engages and to resist torsional forces in the distal inferior direction at the flange opening of rear chassis 508. The receiving flange 594 may comprise an axial wall 596 and a proximal wall 598, and optionally a rotational stop side wall 588. This stop side wall may be provided to limit the amount of handle 524 rotation to a desired amount, and/or to avoid inadvertent dispensing of the syringe material during priming or set up.

Referring now to FIGS. 79A and 79D, the injection control device 500 is shown in its initial configuration, with the chassis 506 in the distal position and the handle 524 in an unlocked position. At the distal position, the follower pin of the front chassis 510 is located at the first end of the spiral recess 520 that is farthest from the axle 518. The syringe is then prepared and then engaged to the device 500, with the syringe body flange placed into the syringe body cavity and the plunger flange placed into the plunger cavity. To the extent the prepared syringe is already attached to a needle or cannula already inserted into a location in the body, the device 500 is manipulated onto the syringe while holding the syringe in place.

Once engaged, the handle 524 is rotated counterclockwise into engagement with the handle lock, until the handle 524 is stopped by the stop wall of the receiving flange 594. This rotation causes the plunger attachment head 526 of the plunger adjustment assembly 522 to be displaced distally, as depicted in FIGS. 79B and 79E, while the chassis 506 remains stationary. This results in the depression or actuation of the plunger relative to the syringe body, and the expression of a limited amount of the syringe contents and/or evacuation of any air bubbles or deadspace within the syringe contents.

When ready to withdraw the syringe and the attached needle or cannula, the primary handle 502 is held to stabilize the position of the device 500 while the plunger handle 524 is grasped and pulled proximally to move the chassis 506 proximally. This movement causes the spiral cam gear 514 to rotate proximally along the rack 516, thereby rotating the spiral recess 520 such that the follower pin 518 is pushed closer to the axle 518 of the spiral cam gear 514. This in turn causes the greater relative displacement of the front chassis 510 toward the rear chassis 508, thereby dispensing a uniform amount of syringe contents per unit of displacement distance of the chassis 506, regardless of variations in the speed or rate of chassis 506 movement. Referring to FIG. 79C and 79F, the chassis 506 is withdrawn until the chassis bracket 540 abuts the stop flange structure 536 of the elongate body 504, and where the follower pin 518 abuts against the second end 564 of the spiral recess 520.

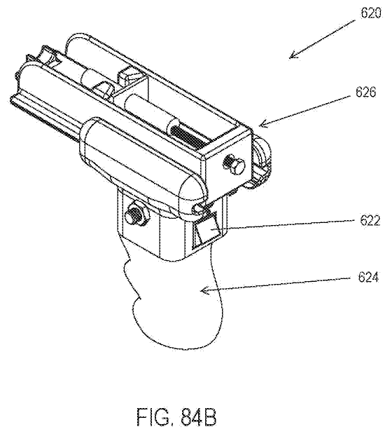

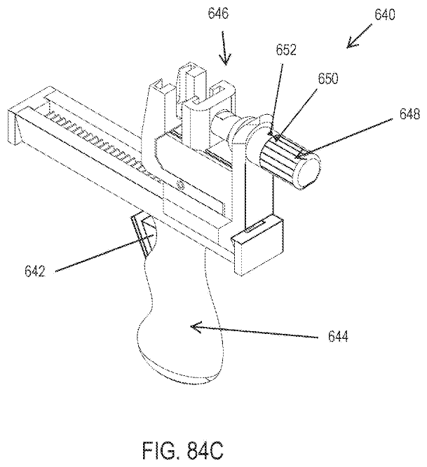

FIGS. 84A to 84C depict variants of the injection control devices 100, 300, 500 wherein, instead or in addition to the manual withdrawal of the syringe and needle/cannula, a motor is provided to actuate the gear of the device. In the example depicted in FIG. 84A, the powered injection control device 600 has a similar mechanism as injection control device 100 depicted in FIGS. 1 to 46. The manual handle, however, has been omitted from the top chassis 602 and a button actuator 604 is included at the back of the top chassis 602. Upon activation of the actuator 604, a motor (not shown) coupled to the gear of the device 600 is activated to rotate the gear and displace the chassis 602 in the proximal direction. A battery and corresponding wiring or circuitry are included chassis 602, but a person of skill in the art will understand that the actuator and battery may also located in the lower chassis 606, with power provided between the top and lower chassis view flexible wiring or flexible circuit board. The motor may be any of a variety of DC motors, and additional gearing may be included to provide a low speed, high torque assembly to move the top chassis 602. The device 600 may be configured to turn on and off the motor based on the user's activation of the actuator 604, so that the displacement of the chassis 602 may be stopped and started as desired by the user. The actuator 604 may be biased to the off position via a spring, so that upon release of the actuator 604, power to the motor is stopped. The actuator 604 may be a button, rocker switch, lever, touch sensor, slider, knob or other actuator known in the art. In other variations, additional circuitry may be provided so that upon activation of the actuator the motor may be activated to complete the entire withdrawal of the syringe regardless of further manipulation of the actuator 604. Strain sensors and/or accelerometers may be also be included to automatically stop the withdrawal of the syringe if excessive movement of the device 600 or resistance to withdrawal is detected.

FIG. 84B depicts another exemplary powered injection control device 620 that has a similar mechanism to injection control device 300 in FIGS. 47A to 75B. A button actuator 622 is provided upper rear surface of the handle 624 to activate the motor (not shown). The device 620 may be actuated by the thumb of the user's gripping hand or by the user's non-gripping hand, but in other variations, the actuator may be provided at the index finger position of the handle 624. The motor and battery (not shown) may be housed in the handle 624 of the device 620, and upon actuation, the motor rotates the gear (not shown) to cause proximal displacement of the chassis assembly 626. The axle of the motor may be coupled to the axle of the gear, or may be coupled to a motor gear that in turn is in a mechanical linkage with the gear that interfaces with the holder gear rack of the chassis assembly 626. Other features and further variations of the device 620 include those described above for powered injection control device 600.

In FIG. 84C, the exemplary powered injection control device 640 is configured with a similar mechanical mechanism as depicted in injection control device 500 of FIGS. 78A to 83B. A trigger actuator 642 is provided on the upper anterior surface of the handle 644. In some further variations, an additional safety switch, slide or button may be provided and must be actuated before or during trigger actuation 642, to reduce the risk of inadvertent actuation of the device 640 when gripping the device 640. In this particular embodiment, the motor (not shown) may be provided in the chassis 646 while the battery (not shown) and the actuator 642 may be located in the handle 644. In other variations, however, the battery and the actuator may also be located on the chassis 646, e.g. at the proximal end of the priming knob 648. In this particular embodiment, the knob 648 is rotated to prime the syringe plunger, but the handle portion of the knob is not included since the motor is used to move the chassis 646. The knob 648 may comprise surface ridges, indentations, projections or flanges to facilitate gripping and rotation. Indicia 650, 652 may be provided to facilitate the desired amount of priming by the user, where the indicia 650 on the knob 648 is rotated into alignment with the complementary indicia 652 adjacent to the knob 648. In other variations, the knob 648 may be configured with a mechanical stop to avoid excessive rotation beyond the desired or maximum priming amount. Upon depressing the trigger 642, the motor in the chassis 646 is activated to move the chassis 646 proximally, thereby withdrawing the syringe and injecting the syringe contents. Other features and further variations of the device 620 include those described above for powered injection control device 600.

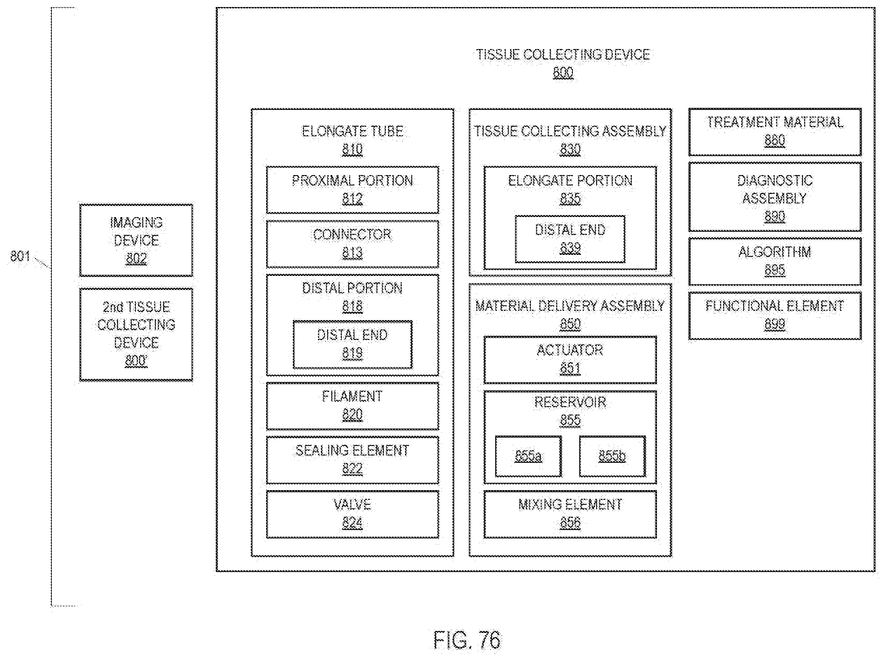

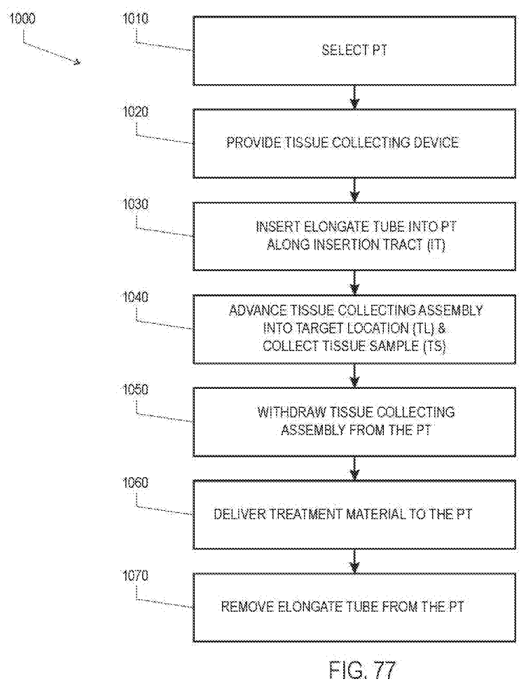

In some embodiments, a device with features similar to those described above with respect to the injection control devices 100, 300, 500, 600, 620, 640 may be used for purposes other than or in addition to controlled injection, such as, for example, tissue collection. Referring now to FIG. 76, a schematic view of a device for collecting tissue from a target location is illustrated, consistent with the present inventive concepts. The tissue collecting device 800 includes various components to allow an operator (e.g. a clinician of the patient) to safely and effectively capture, secure, and/or otherwise collect (“collect” and its derivatives herein) one or more samples of tissue, tissue sample TS. Device 800 can be used to collect tissue from one or more anatomical locations of a patient, target location TL. Device 800 is constructed and arranged to deliver one or more treatment materials 880, to a delivery location DL. In some embodiments, device 800 and its components are used as described in reference to FIG. 77 below.

Device 800 can be constructed and arranged to avoid acute and/or chronic complications (e.g. adverse events) that otherwise may result due to the performance of the tissue collecting procedure, such as those described herein. In some embodiments, device 800 is configured to puncture into an organ (e.g. parenchymal tissue of an organ), and to collect a tissue sample TS (e.g. capture and remove a one or more samples of tissue) for subsequent analyses, such as an analysis where cells are analyzed to determine and/or assess a presence and/or type of cancer cells.

In some embodiments, device 800 comprises an elongate tube 810, a tissue collecting assembly 830, and a material delivery assembly 850, each as shown. In some embodiments, device 800 further comprises one or more materials, e.g., treatment material 880, where material 880 is to be delivered to one or more anatomical locations of the patient, such as to reduce complications associated with the collection of tissue performed by an operator (e.g., a clinician of the patient) when collecting tissue of the patient. In some embodiments, device 800 is of similar construction and arrangement to injection control device 100 and/or injection control device 300, as described herein.

Elongate tube 810 may comprise a proximal portion 812, a distal portion 818, and a distal end 819. Distal portion 818 may be configured to safely and effectively cut through tissue of the patient, such as to cut through parenchyma of an organ. Elongate tube 810 may comprise a rigid tube, such as a tube made of metal, such as steel (e.g., stainless steel). Tube 810 may comprise a tube with at least one flexible portion and at least one rigid portion. Elongate tube 810 may comprise an introducer, such as a standard introducer used as an access device in numerous clinical procedures.

In some embodiments, device 800 comprises an elongate filament 820, which can be positioned within elongate tube 810 (e.g., within a lumen of tube 810) as elongate tube 810 is advanced toward the target location TL creating insertion tract IT. After distal end 819 of tube 810 is positioned proximate (e.g., near, on the surface of, and/or within) target location TL, filament 820 can be removed from tube 810 (e.g., and replaced with tissue collecting assembly 830).

In some embodiments, elongate tube 810 comprises one or more connectors, e.g., connector 813 shown, such as one or more connectors that fluidly attach to tube 810 (e.g., fluidly attach to one or more lumens within tube 810). As one example, connector 813 may include a Luer connector or other conventional fastening mechanism. Connector 813 may be configured to fluidly attach to material delivery assembly 850, such as when treatment material 880 is delivered from assembly 850, through connector 813 and distal end 819, and into the patient (e.g., delivered while tube 810 is stationary and/or being removed from the patient).

Tissue collecting assembly 830 may comprise an elongate portion, portion 835, which may include a distal end 839 that is configured to slidingly pass through one or more lumens of elongate tube 810 such that distal end 839 extends beyond distal end 819 of elongate tube 810. Distal end 839 may comprise a sharpened distal end which can both puncture tissue and collect tissue sample TS. Tissue collecting assembly 830 may comprise a biopsy needle. In some embodiments, at least distal end 839 is configured to be rotated, such as a rotation configured to collect tissue sample TS from the target location TL.

Material delivery assembly 850 may be configured to deliver treatment material 880 to the one or more anatomical locations of the patient. Material delivery assembly 850 may comprise one or more reservoirs 855, such as first reservoir 855a and second reservoir 855b shown. In some embodiments, first reservoir 855a stores a first component of treatment material 880 and second reservoir 855b stores a second component of treatment material 880, such as when treatment material 880 includes at least two components that should be mixed at a time near the time of the delivery of material 880 to the patient, or at a defined time prior to the delivery to the patient. For example, treatment material 880 may comprise two components of a two-part adhesive, or a first component that is a glue (e.g. a bio glue) and a second component that is a treatment material such as 90Y or another radioisotope, and/or a chemotherapeutic. For example, in some embodiments, mixing of two components of treatment material 880 is performed at a time within 10 minutes, within 5 minutes, and/or within 2 minutes of the time of the delivery of treatment material 880 to the patient.

In some embodiments, material delivery assembly 850 comprises a component to mix treatment material 880 (e.g. mix two components of treatment material 880 as described herein), such as mixing element 856 shown. In some embodiments, mixing element 856 comprises an elongate tube with a circuitous (e.g. helical) fluid pathway configured to mix two components (e.g. at least two components) of treatment material 880. In some embodiments, mixing element 856 comprises an agitator, such as a motorized agitator, configured to mix treatment material 880.

Material delivery assembly 850 may include a trigger, an actuating surface, and/or other actuator, e.g., actuator 851 shown. Actuator 851 can comprise a control configured to allow an operator (e.g. clinician) to begin, maintain, modify, and/or stop the delivery of treatment material 880 into the patient. Actuator 851 can be configured to, upon operator activation, to cause mixing of treatment material 880 (e.g. cause mixing of a single or multi-component treatment material 880).

In some embodiments, material delivery assembly 850 comprises a syringe, or a fluid delivery pump, such as a syringe and/or a pump. Material delivery assembly 850 may be configured to fluidly attach to elongate tube 810 (e.g. to a lumen of tube 810, such as via connector 813).

Treatment material 880 is configured to be delivered into the patient, such as to reduce the likelihood of an adverse event, as described herein. Treatment material 880 can include one or more therapeutic agents, such as a radioisotope (e.g. 90Y) and/or a chemotherapeutic, such as to treat cancer and/or reduce the likelihood of the spread of cancer (e.g. via insertion tract IT described herein).

In some embodiments, treatment material 880 comprises a glue (e.g. a bio glue), such as a glue comprising the combination of egg white of bovine serum and glutaraldehyde. Treatment material 880 may comprise a two-part adhesive, such as an adhesive that may be configured to cure within 10 minutes, within 5 minutes, and/or within 2 minutes.

In some embodiments, tissue collecting device 830 comprises functional element 899 shown, which can comprise one or more sensors, one or more transducers, and/or one or more other functional elements. Functional element 899 may comprise a power supply, such as a battery and/or other power supply configured to provide power to another functional element 899 and/or an electronic component of device 800 (e.g. diagnostic assembly 890 described herein). Functional element 899 may comprise an electronics module, such as an electronics module comprising a microprocessor and/or other microcontroller, electronic memory, signal processing circuitry, and the like. In some embodiments, functional element 899 may comprise electronic circuitry configured to interface with one or more transducer-based and/or sensor-based additional functional element 899. In some embodiments, functional element 899 may comprise electronic circuitry that includes algorithm 895 and/or within which algorithm 895 performs one or more analyses. Functional element 899 may comprise a mechanical assembly, such as a mechanical linkage that passes through elongate tube 810. Functional assembly 899 can comprise a fluid delivery assembly, such as a pump.

Functional element 899 may comprise at least one transducer, such as a transducer selected from the group consisting of: an audible transducer; a light emitting element; a display; a tactile transducer; a vibrational transducer; a heat generating transducer; a cooling element; and combinations thereof.

Functional element 899 may comprise at least one sensor, such as at least one physiologic sensor. Functional element 899 may comprise one or more physiologic sensors selected from the group consisting of: blood pressure sensor; heart rate sensor; blood flow sensor; EKG sensor; EEG sensor; respiration sensor; blood gas sensor; oxygen sensor; blood glucose sensor; perspiration sensor; tissue temperature sensor; tissue impedance sensor; body position sensor; and combinations thereof. Functional element 899 may comprise one or more sensors selected from the group consisting of: pressure sensor; strain gauge; accelerometer; impedance sensor; electrode; temperature sensor; light sensor; magnetic sensor; viscosity sensor; camera (e.g. visible light camera; infrared camera, ultrasound imager; CT scanner; and/or MRI); and combinations thereof. Each sensor-based functional element 899 can produce one or more signals representative of the parameter being sensed, such as one or more signals that are provided to another component of device 800 for signal analysis and/or other use (e.g. provided to diagnostic assembly 890 and/or algorithm 895, each as described herein).

Functional element 899 may comprise one or more elements positioned on and/or otherwise proximate elongate tube 810, tissue collecting assembly 830, and/or material delivery assembly 850. In some embodiments, functional element 899 comprises one or more sensors positioned on and/or otherwise proximate material delivery assembly 850, such as when functional element 899 comprises a sensor-based element configured to measure a parameter of treatment material 880 (e.g. when treatment material 880 is positioned within reservoir 855 and/or another portion of material delivery assembly 850), such as when tissue collecting device 800 is configured to detect if an undesired state of treatment material 880 exists. For example, when an undesired temperature or viscosity of treatment material 880 exists, tissue collecting device 800 may be configured to enter an alert state in which a transducer-based functional element 899 alerts the operator of device 800 (e.g. a functional element 899 produces a sound, a visible indicator, and/or a tactile sensation). In some embodiments, tissue collecting device 800 is configured to enter an alert state when elongate tube 810 and/or tissue collecting assembly 830 is at an undesired anatomical location.

In some embodiments, tissue collecting device 800 comprises diagnostic assembly 890 shown, which can comprise one or more assemblies configured to perform a diagnostic procedure. Diagnostic assembly 890 may comprise one or more electronic components, a power supply (e.g. a battery), and/or other componentry. Diagnostic assembly 890 can be configured to monitor a parameter of the patient (including the patient's environment) and/or a parameter of tissue collecting device 800, such as to provide diagnostic information to an operator of device 800. Alternatively or additionally, if an undesired patient and/or device 800 condition is detected by diagnostic assembly 890, tissue collecting device 800 can enter an alert state, such as an alert state in which an audible, visual, and/or tactile alert is produced, and/or a state in which the functionality of device 800 is stopped, limited, or otherwise modified. Undesired patient conditions detectable by diagnostic assembly 890 can include, but are not limited to: undesired patient position (e.g. as determined by a functional element 899 comprising a position sensor, accelerometer, and/or camera); undesired heart rate; undesired blood pressure; undesired tissue temperature; undesired blood gas parameter; and/or undesired blood glucose level. Undesired conditions of device 800 detectable by diagnostic assembly 890 can include, but are not limited to: undesired position of device 800 (e.g. undesired position of distal end 839 for collecting tissue sample TS); undesired temperature (e.g. undesired temperature of reservoir 855); undesired state of a valve of device 800 (e.g. valve 824 described herein); and/or a leak condition (e.g. a condition in which treatment material 880, air, and/or other fluid is leaking into and/or out of device 800).

Diagnostic assembly 890 may comprise a timer assembly (not shown), such as a timer assembly configured to alert an operator that a particular time period has elapsed. This alert can be configured to alert the operator (e.g. an audible, visual, and/or tactile alert provided by a transducer-based functional element 899) if treatment material 880 is in an undesired condition, such as if treatment material 880 comprises a glue that is in an unacceptable state for delivery to the patient (e.g. an unacceptable temperature, an undesired viscosity, an unacceptable state of curing, or the like). In some embodiments, diagnostic assembly 890 is configured to detect if the delivery of treatment material 880 is performed within a pre-determined time limit of a particular event, such as within a pre-determined time from mixing of a two-part adhesive, and/or within a pre-determined time of another event (e.g. within a pre-determined time of collecting of tissue sample TS and/or within a pre-determined time of removing a tissue collecting assembly 830 and/or another device 800 component from elongate tube 810).

In some embodiments, diagnostic assembly 890 is configured to diagnose tissue collecting device 800 via data (e.g. signals) produced by one or more sensor-based functional elements 899 as described herein. In some embodiments, diagnostic assembly 890 is configured to monitor the temperature of one or more portions of device 800, such as one or more portions of reservoir 855. In some embodiments, diagnostic assembly 890 is configured to monitor motion of one or more components that slidingly pass through elongate tube 810, such as to identify a condition in which desired motion is not achieved (e.g. tissue collecting assembly 830 and/or material delivery assembly 850 is not sufficiently translating through tube 810). In some embodiments, diagnostic assembly 890 is configured to monitor a pressure of one or more portions of device 800, such as to monitor the pressure within reservoir 855.

Diagnostic assembly 890 can be configured to assess a physiologic parameter of the patient, such as via data (e.g. signals) produced by one or more sensor-based functional elements 899 as described herein. In some embodiments, diagnostic assembly 890 is configured to determine an appropriate time for tissue collecting assembly 830 to collect tissue sample TS, such as at a time when distal end 839 is properly located at the target location TL (e.g. as determined by diagnostic assembly 890), and/or when a patient parameter (e.g. patient body position, respiration cycle and/or heart cycle) is at an acceptable state (e.g. as determined by diagnostic assembly 890) for distal end 839 to collect tissue sample TS.

In some embodiments, tissue collecting device 800 comprises algorithm 895 shown, which can comprise one or more algorithms configured to analyze data, such as data (e.g. signals) produced by one or more sensor-based functional elements 899, as described herein. In some embodiments, diagnostic assembly 890 may comprise algorithm 895.

Algorithm 895 may be configured to analyze physiologic parameters of the patient and/or parameters of device 800 (e.g. either or both based on signals produced by one or more sensor-based functional elements 899). Algorithm 895 may be configured to cause tissue collecting device 800 to enter an alert state if an undesired condition is detected by algorithm 895.

In some embodiments, elongate tube 810 comprises sealing element 822 which may comprise one or more seals configured to provide a seal between elongate tube 810 and a surface, such as the surface of the patient's skin or another tissue surface of the patient (e.g. a surface of an organ of the patient). In some embodiments, sealing element 822 comprises a cuff material, such as a polyester cuff or other flexible material for providing a seal. In some embodiments, sealing element 822 comprises a flexible component circumferentially placed about an outer surface segment of tube 810.

In some embodiments, elongate tube 810 comprises one or more valves 824, which can be positioned within elongate tube 810 (e.g. within a lumen of elongate tube 810). Valve 824 can be configured to limit (e.g., stop or at least resist) flow of fluid within tube 810, such as to prevent undesired flow of liquids and/or gases into and/or out of the patient (e.g., into and/or out of the target location TL). In some embodiments, valve 824 is configured to allow the passage of one or more elongate filaments, such as filament 820, elongate portion 835 of tissue collecting assembly 830, and/or an elongate portion of material delivery assembly 850. In some embodiments, valve 824 may include a valve that allows flow of a component (e.g. a filament) in one direction.

In some embodiments, a system 801 is provided for collecting tissue from one or more anatomical locations of a patient. System 801 may include one or more devices 800 and an imaging device, device 802 as shown. Imaging device may include one, two, or more imaging devices selected from the group including, for example: X-ray; fluoroscope; CT scanner; PET Scanner; MRI; ultrasound imager; OCT imager; or the like; and combinations thereof. Imaging device 802 can be used to position tissue collecting assembly 830 in the patient to collect target tissue, for example, to provide an image such that an operator of device 800 (e.g., a clinician of the patient) can position distal end 839 at a target location TL. In some embodiments, system 801 may comprise two or more devices 800 provided in a kit form (e.g., devices 800 and 800′ shown), such as when different devices 800 have different configurations, such as when device 800 and 800′ comprise elongate tubes 810 with different lengths, such as to safely, effectively, and efficiently reach deeper or shallower target locations TL within a patient. In some embodiments, a first tissue collecting device 800 collects tissue from a first target location TL1, and a second tissue collecting device 800′ collects tissue from a second target location TL2.

Referring now to FIG. 77, a flow chart of a method for collecting tissue from a target location is illustrated, consistent with the present inventive concepts. Method 1000 includes various steps for safely and effectively capturing tissue from one or more anatomical locations of a patient, such as to avoid acute or chronic complications that otherwise may result due to the procedure. Method 1000 is described using device 800 and its components as described in reference to FIG. 76 herein. Alternatively or additionally, method 1000 can be accomplished using injection control device 100 and/or injection control device 300, each as described herein.

In Step 1010, a patient is selected for performance of a tissue collecting procedure according to the present inventive concepts. The patient can comprise a mammal, such as a human. In some embodiments, the patient is selected based on an assumption that a malignant lesion may be present at one or more target locations TL.

In Step 1020, a tissue collecting device is provided, such as device 800 of FIG. 76. In some embodiments, a device 800 is selected from a kit of multiple tissue collecting devices, such as a kit comprising a first tissue collecting device 800 with a first configuration (e.g., a first size), and a second tissue collecting device 800′ with a second configuration (e.g., a second size) that is different than the first configuration (e.g., the first size and the second size are different). For example, the elongate tube 810 of device 800 can be a different length than an elongate tube 810′ of device 800′.

In Step 1030, elongate tube 810 of device 800 is advanced into the patient, along a path through the patient's tissue, insertion tract IT, to a target location TL. In some embodiments, elongate tube 810 comprises a length that passes through the skin of the patient (an “insertable length”) of at least 0.5 mm, and/or no more than 300 mm. The length (e.g., the insertable length) of the elongate tube 810 may be varied based on the target location TL. In some embodiments, elongate tube 810 comprises an insertable length of at least 0.5 mm, and/or no more than 5 mm, such as when target location TL is a location within subcutaneous tissue and/or other skin tissue of the patient. In some embodiments, elongate tube 810 comprises an insertable length of at least 20 mm, and/or no more than 150 mm, such as when target location TL is a location within the brain of the patient. In some embodiments, elongate tube 810 comprises an insertable length of at least 0.5 mm, and/or no more than 10 mm, such as when target location TL is a location within the thyroid of the patient. In some embodiments, elongate tube 810 comprises an insertable length of at least 0.5 mm, and/or no more than 10 mm, such as when target location TL is a location within the neck of the patient. In some embodiments, elongate tube 810 comprises an insertable length of at least 20 mm, and/or no more than 150 mm, such as when target location TL is a location within a lung of the patient. In some embodiments, elongate tube 810 comprises an insertable length of at least 20 mm, and/or no more than 150 mm, such as when target location TL is a location within the heart of the patient. In some embodiments, elongate tube 810 comprises an insertable length of at least 10 mm, and/or no more than 200 mm, such as when target location TL is a location within a breast of the patient. In some embodiments, elongate tube 810 comprises an insertable length of at least 30 mm, and/or no more than 200 mm, such as when target location TL is a location within the liver of the patient. In some embodiments, elongate tube 810 comprises an insertable length of at least 50 mm, and/or no more than 200 mm, such as when target location TL is a location within the retroperitoneum of the patient. In some embodiments, elongate tube 810 comprises an insertable length of at least 50 mm, and/or no more than 300 mm, such as when target location TL is a location within an intestine of the patient. In some embodiments, elongate tube 810 comprises an insertable length of at least 20 mm, and/or no more than 250 mm, such as when target location TL is a location within a bone of the patient.

In some embodiments, elongate tube 810 is advanced through the skin of a patient (e.g. via a small skin incision) in a percutaneous procedure. Alternatively, elongate tube 810 can be directly advanced into tissue below the skin surface (e.g. directly into an organ), such as when advanced through an open surgical site and/or through a device that provides access to a location within a patient (e.g. a laparoscopic port and/or an endoscope).

In some embodiments, elongate tube 810 is advanced under image-based guidance, such as when elongate tube 810 is advanced using: CT guidance; fluoroscopic guidance; X-ray guidance; ultrasound image guidance; MRI guidance; PET scan guidance; and/or visible camera guidance.

In some embodiments, device 800 comprises a spindle or other elongate filament, such as filament 820 described herein. Filament 820 can be positioned within elongate tube 810 (e.g. within a lumen of tube 810) during advancement of tube 810 to the target location TL, and then removed after distal end 819 of elongate tube 810 is positioned proximate the target location TL (e.g. when the tissue collecting assembly 830 is positioned within elongate tube 810 (e.g. within a lumen of tube 810) after filament 820 is removed.

Target location TL can comprise a location including tumor tissue (e.g. known or suspected of having tumor tissue). Target location TL can comprise a location including tissue within a lung of the patient (e.g. when one or more steps of method 1000 are performed within the lung in an inflated condition and/or in a deflated condition). In some embodiments, target location TL comprises an anatomical location of the patient selected from the group consisting of: organ tissue; lung tissue; liver tissue; brain tissue; breast tissue; intestinal tissue; skin tissue; thyroid tissue; tissue of the neck; heart tissue; tissue of the retroperitoneum; bone tissue; lymphatic tissue; laryngeal tissue; or the like; and combinations thereof.

In Step 1040, elongate portion 835 of tissue collective assembly 830 is advanced through elongate tube 810 (e.g. through a lumen of tube 810) and into a target location of the patient's anatomy. Once in the target location, tissue sample TS is collected by the distal end 839 of the elongate portion 835.

In Step 1050, the tissue collecting assembly 830 is withdrawn from the patient (e.g. with tissue sample TS located within assembly 830).

In Step 1060, treatment material 880 is delivered to delivery location DL by material delivery assembly 850. In some embodiments, delivery location DL comprises at least the insertion tract IT formed in Step 1030. In some embodiments, the delivery location DL comprises one or more locations proximate the location of the target tissue prior to the performance of Step 1040. The delivery location DL can comprise both the insertion tract formed in Step 1030 and one or more locations proximate the location of the target tissue prior to the performance of Step 1040. In some embodiments, the delivery location DL comprises the location of a tunnel or space formed in tissue by at least one of: insertion of the elongate tube; manipulation of the elongate tube; insertion of the tissue collecting assembly; and/or manipulation of the tissue collecting assembly; or the like.

In some embodiments, at least a portion of Step 1060 (e.g. at least a portion of the delivery of treatment material 880) is performed prior to, during, and/or after the performance of Step 1050 (e.g. at least during and after Step 1050). In some embodiments, Step 1060 (e.g. at least a portion of Step 1060) is performed within a particular elapsed time (a “time limit” herein) from the completion of Step 1050 or other particular step of Method 1000, such as within a time limit of no more than 1 hour, 30 minutes, 15 minutes, 10 minutes, and/or 5 minutes (e.g. where device 800 is configured to provide an alert if the time limit has been exceeded and Step 1060 has not been completed or at least initiated).

In some embodiments, at least a portion of Step 1060 is performed prior to, during, and/or after the performance of Step 1070 (e.g. at least during and after Step 1070). In some embodiments, Step 1060 (e.g. at least a portion of Step 1060) is performed within a particular elapsed time (a “time limit” herein) from the completion of Step 1070 or other particular step of Method 1000, such as within a time limit of no more than 1 hour, 30 minutes, 15 minutes, 10 minutes, and/or 5 minutes (e.g. where device 800 is configured to provide an alert if the time limit has been exceeded and Step 1060 has not been completed or at least initiated).

In some embodiments, treatment material 880 is delivered to the delivery location DL via a distal end of material delivery assembly 850. In some embodiments, treatment material 880 is delivered to the delivery location DL via distal end 819 of elongate tube 810 (e.g. when material delivery assembly 850 is fluidly attached, such as via connector 813, to a lumen of tube 810).

In some embodiments, treatment material 880 comprises a two-part adhesive, or another material that cures over time, and treatment material 880 is delivered to the patient prior to significant curing occurs (e.g. within 10 minutes, within 5 minutes, and/or within 2 minutes of the mixing of a two-part material that cures once mixing occurs).

In Step 1070, elongate tube 810 is removed from the patient.

Performance of Method 1000 and/or other use of the tissue collecting devices described herein are configured to reduce the likelihood of occurrence of adverse events, such as to reduce the likelihood of one, two, three, or more adverse events selected from the group consisting of: pneumothorax; hemothorax; hemoptysis; embolism; insertion tract seeding (e.g. spread of cancer through insertion tract IT); and combinations thereof. In some embodiments, the likelihood of an adverse event occurring within 48 hours of the performance of Step 1070 is reduced. In some embodiments, the likelihood of an adverse event related to puncturing an organ is reduced.

In some embodiments, a diagnostic procedure is performed prior to performing Step 1020, such as a diagnostic procedure comprising performing a CT scan and/or PET-TAC procedure (e.g. to assess the safest application for performing Method 1000).

Applicant has conducted human clinical studies using the devices and methods of the present inventive concepts. In particular, these clinical studies include a treatment material 880 comprising a glue including the combination of egg white of bovine serum and glutaraldehyde. Pneumothorax (PNX) is the most common complication of percutaneous lung biopsy procedures with an extremely variable reported incidence from 17% to 26.6%. The incidence of PNX requiring chest drainage placement ranges from 1% to 14.2%. Although not unanimously recognized, several factors have been associated with an increased risk of PNX including injury size, injury depth, presence of chronic obstructive pulmonary disease and even operator experience (Winokur et al., 2013). Table 1 below is a list of known risk factors for PNX after standard percutaneous biopsy in the lung.

TABLE 1

| Risk factor | Incidence of Pneumothorax (%) |
|---|---|
| Size <2 cm | 33-60 |
| Depth <4 cm | 14 |
| Experienced radiologist | 17 |
| Inexperienced radiologist | 30 |
| Presence of chronic | 47 |
| obstructive pulmonary disease | |

In general, the Percutaneous Needle Biopsy (ACR) Guidelines for Quality (Improvement Guidelines for Percutaneous Needle Biopsy, Gupta, et al., 2010) report a pneumothorax rate between 12% and 45% and the placement of chest drainage between 2% and 15%.

Hemorrhagic complications are the second most common type of complication of percutaneous lung biopsy with an incidence between 4% and 27%. CT scans show perilesional areas with frosted glass, indicative of bleeding between 27% and 30% of patients. Hemoptysis occurs in about 4% of patients. Reduced sizes of the lesion (less than 2 cm) are associated with more bleeding, as well as penetration (greater than 4 cm) and multiple penetrations of the pleura. Although hemorrhagic complications can be a source of anxiety for the patient, especially in the case of hemoptysis, about 86% of lung bleedings result from minor alveolar hemorrhages and are rarely severe (Winokur et al. 2013).

As shown in Table 2 below, a recent meta-analysis on the complications related to percutaneous lung diagnostic biopsy (Heerink, et. al. 2017) confirmed the listed incidence rates.

TABLE 2

| | Incidence (95% confidence | |
|---|---|---|
| Complication | interval) | |
| Pneumothorax | 18.8% | (14.6-23.9%) |
| Pneumothorax that requires drainage | 4.3% | (2.7-7.0%) |
| Hemoptysis | 1.7% | (0.9-3.1%) |
| Complex complications | 24% | (18.2-30.8%) |
| Major complications | 4.4% | (2.7%-7.0%) |

In methodologies directly or indirectly set forth herein, various steps and operations are described in one possible order of operation but those skilled in the art will recognize the steps and operation may be rearranged, replaced or eliminated without necessarily departing from the spirit and scope of the present embodiments.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the structures disclosed herein, and do not create limitations, particularly as to the position, orientation, or use of such structures. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. An Injection control device, comprising:
a base member, the base member comprising an elongate body and a rack;
a chassis movably coupled to the base member, the chassis comprising:
a rear chassis movably engaged to the base member and configured to engage a plunger;
a front chassis movably engaged to the rear chassis and configured to engage a syringe; and
a spiral cam gear assembly interfaced with the rack of the base member and coupled to the chassis.

2. The device of claim 1, wherein the spiral cam assembly comprises:
a spiral cam gear coupled to the rear chassis, the spiral cam gear comprising a plurality of circumferential teeth, a spiral cam recess, and a rotation axle; and
a follower pin coupled to the front chassis and located in a spiral cam recess of the spiral cam gear.

3. The device of claim 2, wherein the spiral cam recess has a minimum radius and a maximum radius with a radius difference in the range 5 mm to 20 mm.

4. The device of claim 2, wherein the rear chassis comprises at least one slot and the front chassis comprises at least one strut slidably located in the at least one slot.

5. The device of claim 4, wherein the follower pin is attached to the at least one strut.

6. The device of claim 1, wherein the front chassis comprises a syringe cavity configured to engage a syringe.

7. The device of claim 6, wherein the syringe cavity comprises a first opening from which a syringe body of a syringe is configured to extend distally; a second opening from which a plunger is configured to extend proximally, and a third opening configured to removably engage a syringe body flange of a syringe.

8. The device of claim 7, wherein the first opening is a front opening, the second opening is a rear opening, and the third opening is a top opening.

9. The device of claim 1, wherein the rear chassis further comprises a plunger adjustment assembly.

10. The device of claim 9, wherein the plunger adjustment assembly comprises:
a chassis handle movable relative to the rear chassis; and
a plunger engagement structure comprising a plunger cavity and configured to be movable relative to the rear chassis and the chassis handle.

11. The device of claim 10, wherein the plunger cavity comprises a first opening from which a plunger is configured to extend distally, and a second opening from which the plunger is configured to be removably engaged.

12. The device of claim 11, wherein the first opening of the plunger cavity is a front opening, and the second opening of the plunger cavity is a top opening.

13. The device of claim 10, wherein the plunger engagement structure further comprises a plunger engagement head in which the plunger cavity resides, and a plunger engagement body with a helical interface.

14. The device of claim 13, wherein the chassis handle comprises a helical interface complementary to the helical interface of the plunger engagement body.

15. The device of claim 14, wherein the plunger engagement body comprises a helical thread or groove on an outer surface of the plunger engagement body, and the chassis handle further comprises a lumen containing the helical interface of the chassis handle.

16. The device of claim 10, wherein the plunger adjustment assembly further comprises a chassis handle lock extending from the rear chassis and wherein the chassis handle lock is configured to reversibly engage the chassis handle to resist separation of the chassis handle from the rear chassis.

17. The device of claim 16, wherein the plunger adjustment assembly further comprises a chassis handle stop configured to resist further rotation of the chassis handle.

18. The device of claim 10, wherein the plunger engagement structure is slidably engaged to the rear chassis.

19. The device of claim 18, wherein the rear chassis comprises at least one rail and the plunger engagement structure comprises at least one rail attachment that forms a slidable interface with the at least one rail of the rear chassis.

20. The device of claim 19, wherein the at least one rail comprises two elongate grooves and the at least one rail attachment comprises two projections that have complementary mechanical interfit with the two elongate grooves to resist separation of the rear chassis and the plunger engagement structure.

21. The device of claim 1, further comprising a main handle projecting from the base member.

22. The device of claim 1, wherein the base member comprises a longitudinal recess and the rack is located in the longitudinal recess.

23. The device of claim 1, wherein the chassis comprises a bracket engaged to the base member and configured to resist separation of the chassis from the base member.

24-181. (canceled)
